(19) United States
(12) Patent Application Publication
(10) Pub. No.: US 2025/0382605 A1
(43) Pub. Date: Dec. 18, 2025

(54) MAGNETIC NANOPARTICLES FOR SAMPLE SEPARATION

(71) Applicant: Agilent Technologies, Inc., Santa Clara, CA (US)

(72) Inventors: Jeffrey Carl Braman, Carlsbad, CA (US); Natalia Novoradovskaya, San Diego, CA (US); David Long, Glen Mills, PA (US); Bruce Richter, Wilmington, DE (US); Derick Lucas, Wilmington, DE (US)

(21) Appl. No.: 18/697,401

(22) PCT Filed: Sep. 30, 2022
(86) PCT No.: PCT/US2022/077402

§ 371 Date: May 8, 2025

Related U.S. Application Data
(60) Provisional application No. PCT/US21/52974, filed on Sep. 30, 2021.

(51) Int. Cl. C12N 15/10; C12Q 1/6806

(52) U.S. Cl. CPC C12N 15/1013; C12Q 1/6806

(57) ABSTRACT

Silica-coated magnetic nanoparticles with greater ability to remain dispersed, and methods of making and using silica-coated magnetic nanoparticles, including incubation of an aqueous dispersion of newly made particles at room temperature for a time period resulting in clusters of silica-coated magnetic nanoparticles, the resulting clusters being of an optimal size for viral RNA extraction from human clinical samples. The magnetic nanoparticles comprise a core and a coating, where the core comprises Fe₃O₄ or other magnetic material and the coating has a thickness of from about 1.5 nm to about 2 nm. The magnetic nanoparticles are useful for preparing nucleic acids for analysis, by separating nucleic acids from other components and by normalizing nucleic acid concentrations.

### CROSS-REFERENCE TO RELATED APPLICATIONS

This international application claims priority from International Application No. PCT/US2021/052974, filed on Sep. 30, 2021 with the U.S. Patent and Trademark Office as the Receiving Office, which is incorporated herein by reference in its entirety.

### FIELD OF THE INVENTION

The present invention relates to a silica-coated magnetic nanoparticles (NP), compositions comprising such magnetic nanoparticles, and methods of separating analytes from biological samples using such magnetic nanoparticles and compositions.

### BACKGROUND OF THE INVENTION

Magnetic particles possessing a variety of surface modifications are used to separate molecules from biological samples for further analysis and processing. The molecules to be analyzed are separated from other components in the sample by inducing the binding of desired molecules to magnetic particles, applying an external magnetic field to capture molecules-bound particles from the rest of the sample, washing the particles to remove contaminants and finally recovery of the desired molecules using water or low ionic strength buffer.

Magnetic particles used for separation of biomolecules usually possess at their core magnetic iron oxide. Magnetic iron oxide particles are generally present as magnetite (Fe₃O₄), maghemite (γ-Fe₂O₃) or hematite (Fe₂O₃). The magnetic particles can be coated with silica, for example, and assume a wide particle size distribution from tens to hundreds of nanometers to multiple microns. Many synthetic methods for creating silica coated magnetic particles result in materials with poorly defined (uneven) silica surfaces and/or produce large aggregates that settle out of suspension making them difficult to process. As a result, particles mixing prior to and during a protocol is required to ensure maximum biomolecules binding and recovery of high purity material.

More specifically, silica coated magnetic particles can be used to isolate nucleic acids from a biological sample. The first example of capturing nucleic acids from a sample using silica was demonstrated by Vogelstein [Vogelstein, B. and Gillespie, D., Proc. Nalt. Acad. Sci. USA, 76 (2) 615-619, 1979]. He discovered that nucleic acids would bind tenaciously to silica glass in the presence of a chaotropic salt. Bound nucleic acids could be washed with aqueous organic solvent to remove contaminants while the nucleic acids remained bound to the silica. Nucleic acids were subsequently released from the silica when exposed to low ionic strength buffer. Developing a reproducible synthetic method for creating very or optimally small silica coated “nanoparticles” would allow nucleic acids to be captured on silica surfaces, washed free of contaminants and then eluted in low ionic strength buffer without the attendant problem of agglomeration and subsequent settling.

Nanoparticles (NP) described above could be used for a variety of nucleic acid purification protocols. For example, a diagnostic method could be developed relying on enrichment of RNA from SARS-COV-2 virus, the etiological agent of Covid-19 illness. The resulting RNA could be detected by quantitative reverse transcription polymerase chain reaction (QRT-PCR). A wide variety of other viral and bacterial infectious agents such as Flu-A, Flu-B and Legionella (causing legionellosis), respectively, could be detected using a variety of analytic tools if provided with highly purified NP-based nucleic acids.

Additionally, Next-Generation Sequencing (NGS) protocols could be streamlined, resulting in higher sample throughput by using NP to adjust each NGS library to the same specific target concentration, referred to as “normalization”. Normalization of multiple variable concentration NGS libraries allows them to be pooled prior to loading onto the sequencing instrument. This process allows multiple NGS libraries to be sequenced in one sequencing run, maximizing data collection and minimizing sequencing instrument costs.

There are several existing approaches to normalize variable concentration NGS libraries. These include nucleic acid quantification by spectrophotometry, fluorimetry, quantitative PCR, or electrophoresis followed by calculation of desired concentrations and then dilution of all samples to a normalized concentration. Other approaches for nucleic acid normalization include kits sold by Corning Life Sciences and others. The Corning “AxyPrep Mag PCR Normalizer” protocol states that 10 μL of bead mixture has a binding capacity of 200 ng of input library DNA. The protocol advises that “To minimize the variability within each data point, the input DNA must be at least 3-4 times higher than the desired DNA quantity. For example, if your desired elution DNA concentration is 2 ng/μL, then your sample input must be at least 8 ng/μL”. Achieving these quantities of library DNA, e.g. 400 ng DNA from a 50 μL library recovery volume or 200 nM concentration, in a sequencing project is very difficult. This is especially true if the DNA used for NGS library synthesis is of poor quality such as DNA characteristically recovered from formalin-fixed paraffin embedded (FFPE) samples.

Agilent Technologies, Inc. (Santa Clara, CA) currently offers kits for creating Next Generation Sequencing libraries such as the “SureSelectXT Target Enrichment System”. The SureSelectXT methods create libraries with total non-normalized DNA quantities ranging from 10 nM to approximately 50 nM. These DNA quantities are presumed to be too low to enable use of the AxyPrep Mag PCR Normalizer kit.

The use of nucleic acid-based diagnostics and sequencing in research and medical diagnostics is growing, but preparation of nucleic acids prior to analysis is a significant cost component of nucleic acid analytical techniques such as real-time PCR, nucleic acid sequencing, and hybridization testing. Sample preparation delays test results and limits the ability to run these assays to laboratories with well trained personnel. Current sample preparation processes are laborious, time consuming and require laboratory capability.

There remains a need for magnetic nanoparticles and compositions that simplify and reliably produce purified nucleic acids from a wide variety of sample types for diagnostic and research applications.

### SUMMARY OF THE INVENTION

As one aspect of the present technology, a stable aqueous dispersion of magnetic particles is provided. Each of the individual magnetic nanoparticles is comprised of a magnetic core and a homogeneous conformal coating surrounding the core. The homogeneous conformal coating has a thickness from about 1.5 nm to about 2 nm. The average total diameter of an individual homogeneous conformal coated magnetic nanoparticle is about 10 nm to about 20 nm. In some examples, small clusters of magnetic nanoparticles form having an average cluster size in the dispersion from about 50 to about 190 nm. In some examples, the clusters have an average cluster size in the dispersion from about 10 to about 1000 nm, for example, about 300 nm.

As another aspect of the present technology, methods of making silica-coated magnetic nanoparticles are provided. Fe₃O₄ nanoparticles are provided in an alcohol mixture, and a silicate solution is added while sonicating the mixture. The thickness of the silica coating on the magnetic nanoparticles may be adjusted by changing the amount of silicate added.

In some examples where Fe₃O₄ particles are made using coprecipitation, the present method excludes oxygen during the Fe₃O₄ coprecipitation reaction to minimize/eliminate the formation of unwanted Fe₂O₃. In some examples, the coating thickness is selected to (1) protect the Fe₃O₄ core from further oxidation to Fe₂O₃, (2) fully cover the magnetic core, and (3) alter the surface chemistry of the resulting core-shell particle to include a high density of silanol groups (Si—OH) rendering the final particle more susceptible to the binding of nucleic acids. It has been found that coating thickness and surface area are important parameters regarding use and efficiency of the present magnetic particles for purification of nucleic acids from biological samples.

In another aspect, methods are provided for maintaining an aqueous dispersion of the nanoparticles described herein for a clustering period (“aged” NP) resulting in the formation of stable and optimally sized clusters. For example, the nanoparticles can be maintained at room temperature or between 20° C. and 30° C. during the clustering period, which may be at least one month, alternatively at least two, three, four, five, six, seven or eight months, whereby individual magnetic nanoparticles form clusters with average cluster sizes (e.g., diameters) of a selected value or range. For example, the average cluster size can be at least about 200 nm, alternatively at least about 220 nm, alternatively at least about 235 nm, alternatively at least about 250 nm, alternatively at least about 260 nm and/or the average cluster size can be at most about 400 nm, alternatively at most about 370 nm, alternatively at most about 350 nm, alternatively at most about 320 nm, alternatively at most about 300 nm, alternatively at most about 290 nm. It is expressly contemplated that any of the foregoing minimums and maximums can be combined to form a selected range, such as, for example, a range of from about 200 nm to about 400 nm, alternatively from about 250 nm to about 350 nm. Enhanced detection of nucleic acids in human clinical samples is achieved using aged NP compared to “unaged” NP. In some embodiments, the aged NP form clusters having an average cluster size of about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, or about 320 nm; it is contemplated that any of these values may be combined to form a range. It is further contemplated and hereby stated that each the foregoing approximate values are also a disclosure of the specific values.

In another aspect, methods are provided for preparing nucleic acids for analysis. The methods comprise the steps of combining a sample comprising nucleic acids with a binding medium and a magnetic nanoparticle composition as described herein in a vessel, binding of the nucleic acids to the coating of the magnetic nanoparticles, separating the nanoparticles from liquid within the vessel by use of a magnetic field, removing the liquid from the vessel and retaining the nucleic acid-bound magnetic nanoparticles within the vessel, contacting the nucleic acid-bound magnetic nanoparticles with a washing solution to remove contaminants while the magnetic nanoparticles remain attracted to the magnet or are dispersed and then re-attracted by the magnetic field followed by removal of the wash solution, and finally adding an elution medium and collecting released nucleic acids from the magnetic nanoparticles. The nucleic acids are removed from the vessel thereby providing a nucleic acid preparation.

As another aspect of the present invention, methods are provided for obtaining RNA from a biological sample and preparing the RNA for analysis. The methods comprise placing a biological sample comprising RNA in a vessel, wherein the biological sample is a cell or a virus. The biological sample is contacted with guanidine thiocyanate (GTC) and Proteinase K to form a sample mixture, which is incubated at an elevated temperature for a sample preparation period. Neat alcohol and a magnetic nanoparticle composition (embodiments of which are described herein) are added to the sample mixture in the vessel. The methods also comprise mixing the sample mixture and the magnetic nanoparticles and incubating for a second incubation period and then applying a magnetic force to separate the magnetic nanoparticles from a supernatant within the vessel. The supernatant is removed from the vessel without disturbing the separated magnetic nanoparticles, which are washed one or more times with an alcohol solution. The separated magnetic nanoparticles are dried, and an elution medium is added to the vessel and mixed with the magnetic nanoparticles. After an elution period in which RNA is eluted from the magnetic nanoparticles to form an eluate, the eluate is removed from the vessel.

These and other features and advantages of the present methods and compositions will be apparent from the following detailed description, in conjunction with the appended claims.

### BRIEF DESCRIPTION OF THE DRAWINGS

FIGS. 1A and 1B are illustrations of an embodiment of the preparation of silica-coated magnetic nanoparticles according to the present technology.

FIGS. 2A and 2B are electron micrographs showing individual silica-coated Fe₃O₄ nanoparticles and typical cluster size formed by the unaged particles in aqueous dispersions.

FIG. 2C shows laser diffraction data for silica-coated magnetic nanoparticles according to the present technology. FIGS. 2D to 2I are TEM images for silica-coated magnetic nanoparticles prepared in Example 1.

FIG. 3 shows the full spectrum of the silica-coated magnetic nanoparticles by FT-IR spectroscopy. Clearly seen is the Fe—O stretching vibration at about 580 cm−1 and the Si—O—Si stretching vibration at about 1100 cm−1.

FIG. 4 is a series of IR spectra emphasizing the increase of the Si—O to Fe—O peak ratio as an increasing volume of TEOS was used for the silica coating process (0.5 ml to 2.5 ml). The higher Si—O to Fe—O peak ratio signals the formation of thicker silica shells on the Fe₃O₄ nanoparticle core.

FIG. 5 is a graph illustrating that the thickness of the silica coating increases linearly with increasing TEOS amounts.

FIG. 6 is a scanning electron microscopy (SEM) image of silica-coated magnetic nanoparticle according to the present technology, at a magnification of 250,000×. The particles were made with 1.5 ml TEOS and sonication.

FIG. 7 is a transmission electron microscopy (TEM) image of silica-coated magnetic nanoparticle according to the present technology. Typical Fe₃O₄ cores range from 7 to 10 nm. The average silica coating thickness on the particles using 1.5 ml of TEOS was about 1.6 nm. Prominent Fe₃O₄ crystalline lattice visible inside core indicates the lack of oxidation during synthesis.

FIGS. 8A through 8F show Diagnostic Quantitative Reverse Transcription Polymerase Chain Reaction (QRT-PCR) results. Using manual (FIGS. 8A to 8C) and automated (FIGS. 8D to 8F) protocols and multiple lots of magnetic silica nanoparticles, viral RNA was recovered from a sample and analyzed by diagnostic QRT-PCR.

FIG. 9 shows NGS related “Normalization” results. The data presented is electrophoresis and next generation sequencing data metrics verification that normalization, as defined below, is accomplished.

FIG. 10 provides photographs of the magnetic separation of silica-coated magnetic nanoparticles from a stable suspension using a small magnet.

FIGS. 11A to 11K shows results from the normalization of NGS libraries, analyzed by various parameters.

FIGS. 12A, 12B, 13A and 13B illustrate that clusters of nanoparticles increased in size due to aging.

The present teachings are best understood from the following detailed description when read with the accompanying drawing figures. The features are not necessarily drawn to scale.

### DETAILED DESCRIPTION

The silica-coated magnetic nanoparticles (also abbreviated as “NP” herein) described by the present disclosure are surprisingly effective for separating nucleic acids from a sample. The silica coating provides a hydrophilic substrate, greatly reducing non-specific binding in important applications, such as purification of nucleic acids.

In some embodiments, the present technology is used for enrichment of RNA obtained from cellular or viral samples as part of a quantitative reverse transcription polymerase chain reaction (QRT-PCR) detection assay. By way of example, the present technology can be employed to enrich RNA from SARS-COV-2 or other viral pathogens prior to detecting the RNA by QRT-PCR. In some embodiments, the present technology is used for enrichment of human RNA and human and bacterial DNA. For example, the present technology may be used to enrich and detect DNA from respiratory pathogens such as Legionella (Legionnaires' disease pathogen). In some embodiments, the present technology is used for normalization of a DNA library prior to Next Generation Sequencing (NGS). In some embodiments, the present technology is used for normalization of a DNA or RNA sample prior to analysis of the sample.

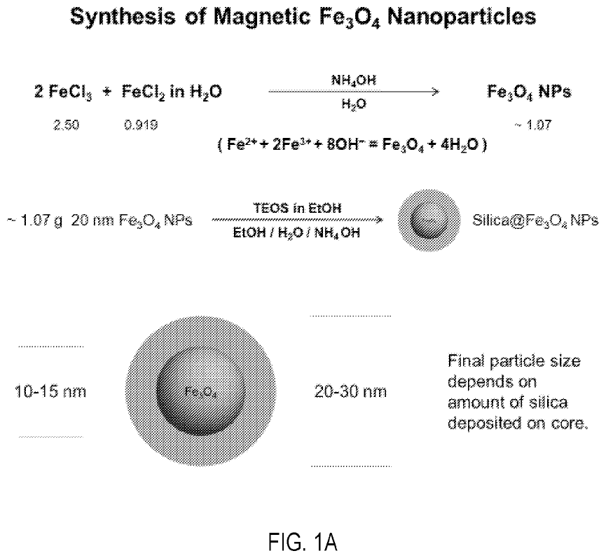

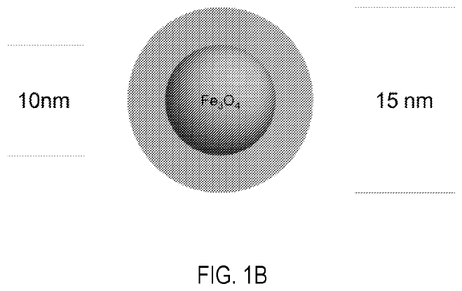

Referring to FIG. 1A, the present technology provides a magnetic nanoparticle 100 comprising a core 10 and a homogeneous conformal coating 20 on the core 10 that substantially encapsulates the core 10. In one example, the coating 20 completely encapsulates the core 10. The core 10 can be made of a metal, an alloy, or a metal oxide and the coating can be made of silica. In an example, the core 10 can be Fe₃O₄ and the coating comprises silica. In an example, the silica coating is substantially non-porous. The ratio of the core diameter to the coating thickness and the characteristics of the coating surface may be such that a plurality of the magnetic nanoparticle 100 aggregate in a manner that allows an analyte in a sample solution to optimally bind on the surface of the coating of the magnetic nanoparticles 100. The magnetic nanoparticles 100 having the analytes bound on its coated surface can then be rapidly removed from the sample solution by applying a magnetic field to the sample solution having the magnetic nanoparticle 100.

FIG. 1A illustrates an embodiment of the preparation of the present magnetic nanoparticle 100. In one example the magnetic nanoparticle 100 can include a size of from about 9 nm to about 40 nm, such as from about 20 nm to about 30 nm. The core 10 can include a diameter of from about 5 nm to about 20 nm, such as from about 7 nm to about 15 nm or less, for example from about 10 nm to about 15 nm or less, such as about 8.8 nm or less, about 8 nm or less, or about 7.9 nm. The coating 20 substantially encapsulating the core 10. In some examples, the coating 20 can include a thickness of from about 0.5 nm to about 7.5 nm, from about 1 nm to about 2 nm, for example, from about 1.5 nm to about 2 nm. FIG. 1B illustrates the magnetic nanoparticle 100 having a particle size of about 15 nm with a coating thickness of about 2.5 nm.

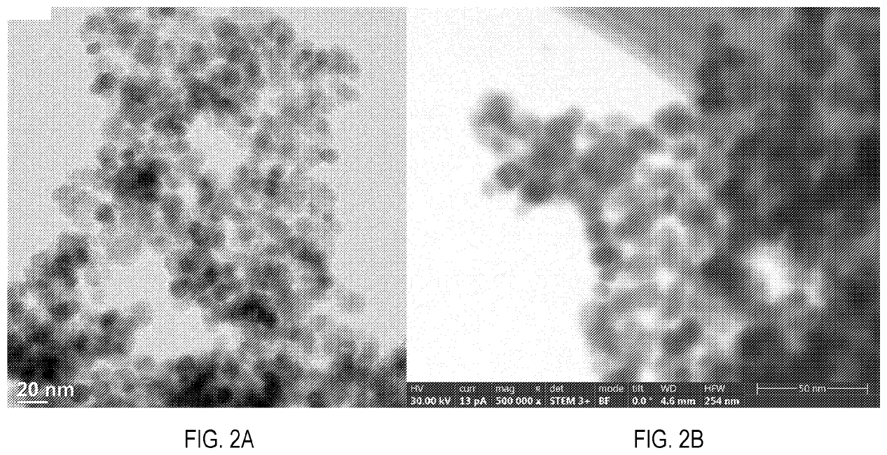

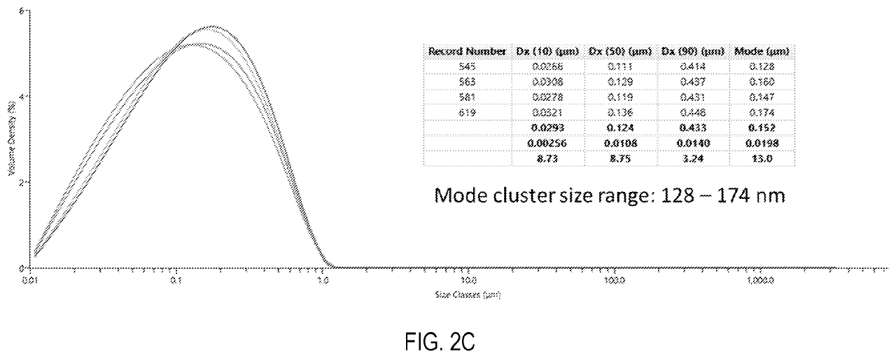

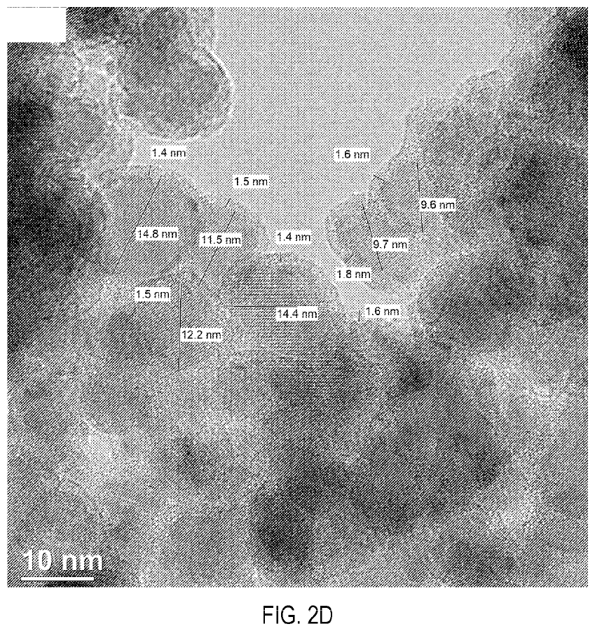

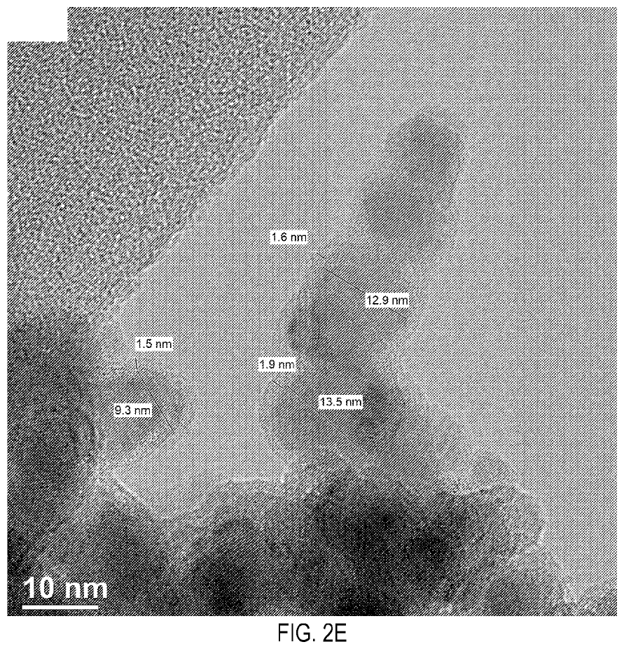

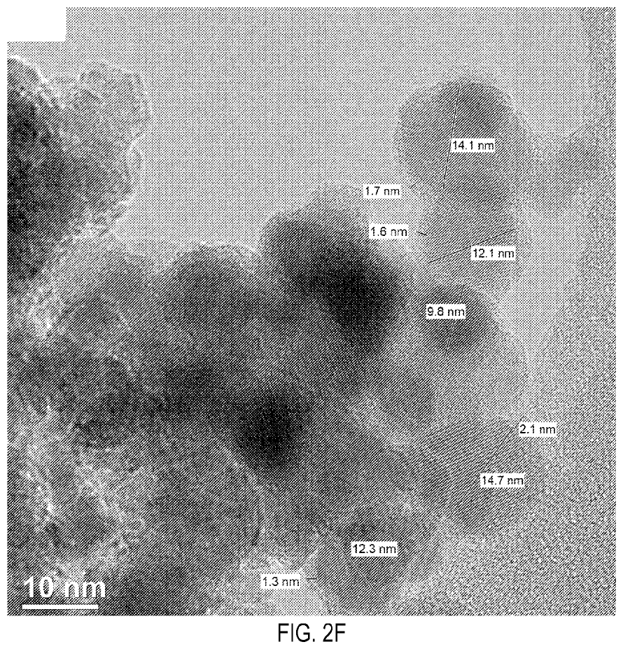

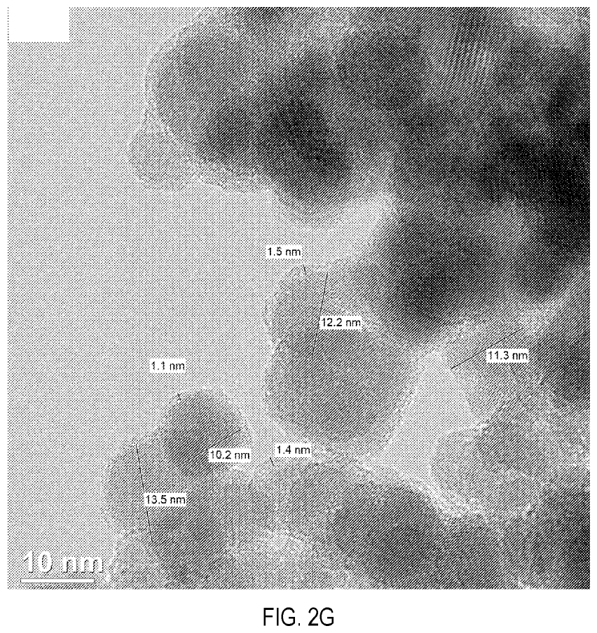

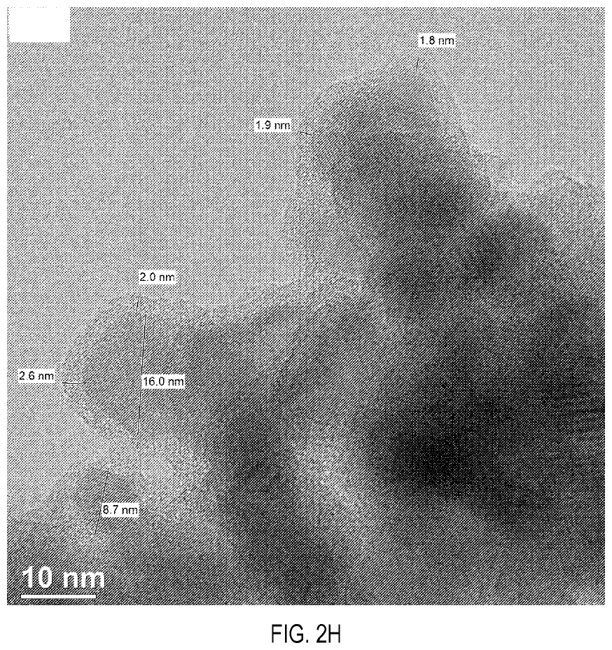

The ratio of the core diameter to the thickness of the silica coating 20, in combination of the characteristics of the silica coating 20 allows the magnetic nanoparticles 100 to aggregate and create clusters as shown in FIGS. 2A and 2B. In an example, the clusters can be from about 10 nm to about 1000 nm, such as from about 20 nm to about 500 nm, from about 100 nm to about 500 nm, from about 200 nm to about 500 nm, from about 250 nm to about 450 nm, from about 275 nm to about 325 nm (e.g., about 300 nm), from about 30 nm to about 400 nm, from about 40 nm to about 300 nm. FIG. 2C shows laser diffraction data measured with a Mastersizer 3000 from Malvern Panalytical, showing the distribution of particle sizes in a typical aqueous dispersion of NP. Individual particles up to clusters approximately 1 micron in diameter are present in the dispersion with the average cluster size being 128-174 nm in diameter. The nanoparticles of this example were “unaged”.

The present NP show high magnetic susceptibility in manual and automated workflows. This is particularly desirable in automated platforms where the timing and efficiency of magnetic particle capture is important, requiring rapid and quantitative particle collection at capture, wash, and elution steps.

In some embodiments, the core of the nanoparticle is mostly or entirely composed of a crystalline lattice of Fe₃O₄. In some embodiments, the core of the nanoparticle is mostly or entirely composed of a single nanoscale crystal of magnetite (Fe₃O₄) or single domain magnetic nanoparticle of Fe₃O₄ that displays paramagnetism.

In some examples, the NP has a Brunauer-Emmett-Teller (BET) surface area of at least 105 m²/g, such as at least from about 110 m²/g to about 180 m²/g or at least from about 110 m²/g to about 130 m²/g. The BET surface area of the NP can be determined by the nitrogen adsorption technique. For example, nitrogen adsorption/desorption isotherms can be measured at liquid nitrogen temperature (−196° C.) using a Micromeritics ASAP2020 volumetric adsorption analyzer for mesoporosity determination.

The silica-coated magnetic nanoparticles described herein can be used directly or functionalized to suit specific application needs. Functional groups such as carboxylate, epoxide, and tosylate can be included in or added to the silica coating, and such functional groups can be a convenient method for covalent functionalization. As further examples, the silica coating can also be modified by incorporating one or more types of organic groups into the silica matrix. Organic groups can covalently functionalize the surface and/or the pores of the silica coating. As another example, polymers can also functionalize the silica surface either by a simple coating or covalently attached.

The coatings of the silica magnetic nanoparticles described herein can include pores that extend to the surface of the magnetic nanoparticle. The coating can be modified with functional groups both inside and outside its pores. In some examples, the coating further comprises a reactive chemical moiety configured to bind an analyte, such as a nucleic acid.

The present technology provides compositions that can include a plurality of the magnetic nanoparticles described herein. The plurality of magnetic nanoparticles have a mean particle size of about 15 nm or less, or about 12 nm or less, or about 11 nm or less. The composition is a stable suspension of the magnetic nanoparticles in a liquid medium, such as water. In some examples, the magnetic nanoparticles are present in the stable suspension at a concentration of from 3 to 30 g/L, or from 10 to 20 g/L, or about 13 g/L.

In some examples, the nanoparticles remain suspended for at least 6 months at a temperature of 25° C., alternatively for at least 9 months. In some examples, the nanoparticles are held for an aggregation period before being used, shipped to a user, or released for general use or a specified use. The aggregation period is selected so that the nanoparticles form clusters of a desired size or character. Exemplary aggregation periods include at least about one month, at least about two months, at least about three months, at least about four months, at least about six months, at least about eight months, or longer; alternatively, exemplary aggregation periods include at most about eighteen months, at most about twelve months, at most about ten months, at most about eight months, at most about six months, at most about five months, or shorter; it is contemplated that any of the foregoing minimums and maximums can be combined to form a range, so long as the minimum is shorter than the maximum. In some examples, the general use is separating an analyte, such as RNA, from a sample. In some examples, the specified use is normalization of a quantity of nucleic acids obtained from a sample.

The present technology also includes packaged stable suspensions of magnetic nanoparticles. The magnetic particles or compositions described herein can be provided in a sealed package, wherein the interior volume of the sealed package is a suspension of nanoparticles in water.

The present technology provides a method of making silica-coated magnetic nanoparticles. The present method for producing silica-coated magnetic nanoparticles includes by synthesizing magnetic iron oxides from aqueous Fe³⁺/Fe²⁺ salt solutions through the addition of a base (such as ammonia or NaOH) under an inert atmosphere at room temperature. Fe₃O₄ particles may be made using coprecipitation. The size, shape, and composition of the magnetic nanoparticles depends on the type of salts used (e.g. chlorides, sulfates, nitrates), the Fe³⁺/Fe²⁺ ratio, the reaction temperature, and pH value. Once the synthetic conditions are fixed, the final characteristics of the magnetite nanoparticles synthesized become fully reproducible. The advantages of this technique in forming Fe₃O₄ nanoparticles include rapid formation of particles, use of inexpensive solvents, magnetic separation for isolation of product, controlled particle size and morphology, reproducible magnetic properties, and the ability to perform synthesis of nanoparticles on larger industrial scales. In some examples, the Fe+ concentration used for the coprecipitation, less than 0.1 M, alternatively less than 0.05 M, alternatively less than 0.02 M, for example 0.01 M.

The methods include preparing an aqueous solution of Fe³⁺ and Fe²⁺ ions in deionized, de-oxygenated water in a Fe³⁺/Fe²⁺ molar ratio of about 2/1. In some examples, the concentration of Fe³⁺ in the aqueous solution is from about 2 to about 50 mM, and the concentration of Fe²⁺ in the aqueous solution is from about 1 to about 25 mM. The aqueous solution can be prepared by dissolving FeCl₃ and FeCl₂ in deionized, de-oxygenated water, though other iron salts may be used as well. The water can be de-oxygenated by any suitable technique, such as by sparging with an inert gas, sonicating, and/or stirring, for a sufficient de-oxygenating period before combining the water with iron salts. The de-oxygenating period will generally be at least two hours, though longer or shorter periods may be employed.

The aqueous solution of Fe³⁺ and Fe²⁺ ions may then be sonicated, and optionally its temperature may be adjusted to about 35° C., such as by heating the aqueous solution from room temperature. A base may be added to the heated aqueous solution under an inert atmosphere. In some examples, suitable bases include ammonia, sodium hydroxide, and others. The base is added in an amount sufficient for formation of Fe₃O₄ cores from the aqueous solution. For example, the base may be added at a 2-fold or greater (e.g., 4-fold or 8-fold) molar excess. As a result of adding a sufficient quantity of base, a mixture is formed which includes Fe₃O₄ nanoparticles and alkaline water. In some examples, the base may be added dropwise to the aqueous solution, optionally while stirring and/or sonicating the aqueous solution.

The mixture is maintained under an inert atmosphere without stirring for a settling period, during which the Fe₃O₄ nanoparticles settle out of the alkaline water. In some examples, the settling period is overnight, or about 12 hours; in other examples, the settling period is at least 4, 6, 8, 12, or 18 hours, and/or no more than 60 hours, 48 hours, 36 hours, 24 hours or 20 hours; the foregoing values can be combined to form a settling period range. After the settling period, most of the alkaline water is removing from the mixture via aspiration. This provides a concentrated mixture of Fe₃O₄ nanoparticles in alkaline water.

A de-oxygenated alcohol is added to the concentrated mixture to form an aqueous alcohol mixture. The aqueous alcohol mixture is stirred and sonicated to suspend the Fe₃O₄ nanoparticles. In some examples, additional base is added to the aqueous alcohol mixture before further processing.

While sonicating and maintaining an inert atmosphere, a silicate solution is added to the aqueous alcohol mixture. The silica coating thickness can be controlled by the amount of the reactants used. The silicate solution may be added dropwise slowly, for example, over a period of from 30 to 90 min. to the aqueous alcohol mixture while stirring and sonicating the mixture. The silicate solution includes tetraethyl orthosilicate (TEOS) and an alcohol. In some examples, the silicate solution is anhydrous.

This mixture is stirred for a coating period under the inert atmosphere, during which a silica coating forms on the Fe₃O₄ nanoparticles. In some examples, the coating period is overnight, or about 12 hours; in other embodiments, the coating period is at least 4, 6, 8, 12, or 18 hours, and/or no more than 60 hours, 48 hours, 36 hours, 24 hours or 20 hours; the foregoing values can be combined to form a coating period range.

The silica-coated Fe₃O₄ nanoparticles are separated from the alcohol aqueous mixture by any suitable separation technique. In some examples, the Fe₃O₄ nanoparticles are separated by magnetic capture from the aqueous alcohol mixture. The silica-coated Fe₃O₄ nanoparticles can be washed with methanol or other alcohol or solvent. The separated silica-coated Fe₃O₄ nanoparticles can be dried, such as through vacuum drying. A powder of the silica-coated Fe₃O₄ nanoparticles is formed by drying. In some examples, the powder comprising silica-coated Fe₃O₄ nanoparticles is formed without milling or mechanically separating the nanoparticles.

The resulting dried nanoparticles may be dispersed into de-oxygenated water to form a composition. In some examples, the composition is a stable suspension. The resulting suspension of particles can be aged for up to 6 months to allow the average cluster size of the nanoparticles to increase from an average of 125 nm to 260 nm.

The present technology also includes methods for preparing nucleic acids for analysis, by separating the nucleic acids from a sample. In some examples, the methods comprise the steps of combining a sample comprising nucleic acids with a binding medium and a magnetic nanoparticle composition as described herein in a vessel. The nucleic acids bind to the coating of the magnetic nanoparticles. After a binding period, the nucleic acid-bound magnetic nanoparticles are separated from the binding medium within the vessel. The nucleic acid-bound magnetic nanoparticles are contacted with a washing solution to remove unwanted contaminants and then contacted with an elution medium, thereby separating the bound nucleic acids from the magnetic nanoparticles. The magnetic nanoparticles are drawn by a magnet and the resulting clarified solution containing the desired nucleic acids are removed from the vessel, thereby providing a nucleic acid preparation.

The magnetic nanoparticles and compositions described herein have many uses, such as for separation of analytes such as small molecules, protein, and/or nucleic acids from biological samples. The magnetic nanoparticles and compositions may be used as carriers for chemical or biological species, including, for example, noble metal particles, small organic or inorganic molecules, DNA, peptides or polypeptides (e.g. antibodies and other proteins), and whole cells. Applications for such carriers may include magnetic resonance imaging (MRI), optical imaging, targeted drug delivery, and cell delivery.

The present technology provides a method for preparing nucleic acids for analysis. The method comprises the steps of combining a sample comprising nucleic acids with a binding medium and a magnetic nanoparticle composition as described herein in a vessel. The nucleic acids bind to the coating of the magnetic nanoparticles. After a binding period, the nucleic acid-bound magnetic nanoparticles are separated from the binding medium within the vessel. In some examples, the nucleic acid-bound magnetic nanoparticles are then washed with a washing medium comprising aqueous alcohol or a variety of water miscible organic solvents, for example acetonitrile, acetone and sulfolane. Most of the liquid (binding medium and/or washing medium) is then removed from the vessel while taking care to retain the nucleic acid-bound magnetic nanoparticles within the vessel. The nucleic acid-bound magnetic nanoparticles are contacted with an elution medium, thereby separating the bound nucleic acids from the magnetic nanoparticles. The nucleic acids are removed from the vessel, thereby providing a nucleic acid preparation.

The coating of the magnetic nanoparticles may comprise a binding moiety configured to selectively bind nucleic acid or another analyte.

The separation of nucleic acids from a sample is illustrative of separating other analytes, such as small molecules such as pharmaceutical agents, proteins or polypeptides, lipids, or other analytes.

The present method may be employed to normalize the quantity of nucleic acids obtained from a sample. In such methods, the sample includes an input quantity of nucleic acids, and the magnetic nanoparticle composition has a binding capacity for an output quantity of nucleic acids. The output quantity is less than the input quantity.

The magnetic nanoparticle composition may have a normalization factor between 0.8 and 1.2, wherein the normalization factor refers to the concentration value of nucleic acids recovered from magnetic silica nanoparticles after exposure to a high concentration of nucleic acids divided by the concentration value of nucleic acids recovered from magnetic silica nanoparticles after exposure to a low concentration of nucleic acids. Ideally for compositions prepared for NGS, the normalization factor will be about 1.0.

The nucleic acid preparation includes nucleic acids having lengths within a predetermined length range. In some examples, the minimum nucleotide length is 50 nucleotides (nt), or 70 nt, or 75 nt, or 80 nt, or 100 nt, or 150 nt, or 200 nt, or 500 nt, or 2,000 nt, or 5,000 nt, or 10,000 nt, or 50,000 nt, or 100,000 nt; the maximum nucleotide may be 2 million nt, or 1 million nt, or 500,000 nt, or 200,000 nt, or 75,000 nt, or 25,000 nt, or 12,500 nt, or 6,000 nt, or 3,000 nt, or 1,500 nt, or 750 nt, or 400 nt, or 250 nt; any of the foregoing minimum and maximum may be combined to form a desired nucleotide length range, so long as the minimum is smaller than the maximum.

The present method may be employed with the input sample includes a low quantity of nucleic acid, for example, less than 1,000 ng, or less than 500 ng, or less than 200 ng, or less than 100 g, or less than 50 ng of nucleic acid.

The present technology provides a method of obtaining RNA from a biological sample and preparing the RNA for analysis. In the method, a biological sample comprising RNA is placed in a vessel. The biological sample may be a cell or a virus.

The method includes contacting the biological sample with guanidine thiocyanate (GTC) and Proteinase K to form a sample mixture, which is incubated at an elevated temperature (such as about 60° C.) for a sample preparation period (such as 10 minutes or less, or about 5 minutes). The GTC and Proteinase K may be added separately or premixed and added together. After the sample preparation period, neat alcohol is added to the sample mixture in the vessel, and one of the magnetic nanoparticle compositions described herein is also added to the sample mixture in the vessel. The sample mixture and the magnetic nanoparticles are mixed and incubated for a second incubation period (such as 10 minutes or less, or about 5 minutes).

After the second incubation period, a magnetic force is applied to separate the magnetic nanoparticles from a supernatant within the vessel. The supernatant is removed from the vessel without disturbing the separating magnetic nanoparticles, which are then washed one or more times with an alcohol solution. Suitable alcohol solutions may comprise 80% v/v or greater of a lower alcohol such as methanol, ethanol, n-propanol, iso-propanol, or a mixture thereof. A substantial variety of non-alcohol organic solvents that are miscible with water are also useful for the washing purpose and are known to those skilled in the art. Several of these include, but are not limited to acetonitrile, acetone and sulfolane. The separated magnetic nanoparticles are dried, and an elution medium is added to the vessel. The magnetic nanoparticles are mixed with the elution medium for an elution period (e.g., less than 10 minutes), after which RNA is eluted from the magnetic nanoparticles to form an eluate. The eluate containing RNA is removed from the vessel, and the RNA may then be subjected to analysis such as quantitative reverse transcription polymerase chain reaction (QRT-PCR) for diagnostic and research purposes or can be modified to serve as a substrate for nucleotide sequencing such as Sanger and NGS sequencing. Alternatively, the removed eluate may be placed in a second vessel which is sealed and stored at a reduced temperature, such as for shipping or later analysis.

In some examples, one, several or all steps of the foregoing methods are performed by an automated instrument.

### Terminology

It is to be understood that the terminology used herein is for purposes of describing particular embodiments only and is not intended to be limiting. The defined terms are in addition to the technical and scientific meanings of the defined terms as commonly understood and accepted in the technical field of the present teachings.

The term “nucleic acid” and “polynucleotide” are used interchangeably herein to describe a polymer of any length, e.g., greater than about 10 bases, greater than about 100 bases, greater than about 500 bases, greater than 1000 bases, greater than 10,000 or more bases, composed of nucleotides, e.g., deoxyribonucleotides or ribonucleotides, or compounds produced synthetically which may hybridize with naturally occurring nucleic acids in a sequence specific manner analogous to that of two naturally occurring nucleic acids, e.g., can participate in Watson-Crick base pairing interactions. Naturally occurring nucleotides include guanine, cytosine, adenine, thymine and uracil (G, C, A, T, and U respectively).

The term “complementary,” “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T/U, G, and C of one sequence is then aligned with a T/U, A, C, and G, respectively, of the other sequence.

The term “duplex” means at least two sequences that are fully or partially complementary undergo Watson-Crick type base pairing among all or most of their nucleotides so that a stable complex is formed. The terms “annealing” and “hybridization” are used interchangeably to mean the formation of a stable duplex.

The terms “hybridization”, and “hybridizing”, in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like.

As used in the specification and appended claims, and in addition to their ordinary meanings, the terms “substantial” or “substantially” mean to within acceptable limits or degree to one having ordinary skill in the art. For example, “substantially cancelled” means that one skilled in the art considers the cancellation to be acceptable.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same.

In the present disclosure, numeric ranges are inclusive of the numbers defining the range. It should be recognized that chemical structures and formula may be elongated or enlarged for illustrative purposes.

Unless defined otherwise, all technical and scientific terms used herein have the same meaning as commonly understood by those working in the fields to which this disclosure pertain.

Before the various embodiments are described, it is to be understood that the teachings of this disclosure are not limited to the particular embodiments described, and as such can, of course, vary. It is also to be understood that the terminology used herein is for the purpose of describing particular embodiments only and is not intended to be limiting since the scope of the present teachings will be limited only by the appended claims.

As disclosed herein, a number of ranges of values are provided. It is understood that each intervening value, to the tenth of the unit of the lower limit, unless the context clearly dictates otherwise, between the upper and lower limits of that range is also specifically disclosed.

Unless defined otherwise, all technical and scientific terms used herein have the same meaning as commonly understood by one of ordinary skill in the art to which this disclosure belongs. Although any methods and materials similar or equivalent to those described herein may also be used in the practice or testing of the present teachings, some exemplary methods and materials are now described.

All patents and publications referred to herein are expressly incorporated by reference.

As used in the specification and appended claims, the terms “a,” “an,” and “the” include both singular and plural referents, unless the context clearly dictates otherwise. Thus, for example, “a moiety” includes one moiety and plural moieties.

### EXAMPLES

### Example 1

In this example, silica-coated magnetic nanoparticles (NP) are made using the method described previously and below. An exemplary procedure to synthesize Fe₃O₄ nanoparticles on the 1 g scale was performed as follows: 400 ml of DI water was extensively de-oxygenated using hard N₂ purging, mechanical stirring, and sonication for 2 hrs. Once de-oxygenated, 2.508 g of FeCl₃ (9.24 mM) and 0.919 g FeCl₂ (4.62 mM) were added under nitrogen and additionally purged/sonicated. Over the course of the sonication, the temperature of the water increased to 35° C. Afterwards, 10 ml of 28% ammonia solution was quickly added (0.18 M, which is approximately a 4 molar excess required for complete conversion of the iron to magnetite) to the rapidly stirring and sonicating solution. The solution became instantly black with nanoparticle precipitate. Sonication was stopped and the mixture was stirred under nitrogen for another 4 hours. Afterwards, the particles were left to sit under nitrogen until the next day without stirring. The nanoparticles settled to the bottom of the flask yielding a clear upper aqueous layer and a black bottom layer consisting of the magnetic Fe₃O₄ nanoparticles.

On the next day the settled Fe₃O₄ nanoparticles were isolated by removing via aspiration a clear upper layer of ammonia/water. The remaining solvent (100 ml) was left in place. Three-hundred ml of de-oxygenated ethanol was then added to the black slurry. The particles were stirred and sonicated for 1 hr. to resuspend the nanoparticles. While sonicating, 1.5 ml of TEOS was mixed with 80 ml of anhydrous ethanol and placed in an addition funnel under a nitrogen purge and fitted to the flask. The flask was purged, then sealed under flowing N₂. To the black nanoparticle suspension was then added 5 ml of additional ammonia solution (28%). The mixture was again sonicated and mechanically stirred for 10 minutes under a nitrogen flow. The TEOS solution was then added dropwise over the period of about 60 min. to the stirring and sonicating nanoparticles. After addition of the TEOS solution, the stirring was continued slowly overnight under N₂.

On the next day, a magnet was used to isolate the nanoparticles to the bottom of the flask. The clear ethanol/water mixture was decanted off the nanoparticles which were held in place by the magnet. The silica-coated Fe₃O₄ nanoparticles were washed with fresh methanol twice and magnetically separated. The nanoparticles were then dried under a stream of nitrogen to a powder, then dispersed into 100 ml of de-oxygenated water. The nanoparticles were sparged extensively with nitrogen and sonicated before sealing under inert atmosphere yielding a 1.3 wt. % solution of nanoparticles in water.

Samples of the silica-coated Fe₃O₄ nanoparticles were isolated as the dry particles in order to perform various analyses. Scanning electron microscopy (SEM) was used to image the particles to approximate the degree of aggregation and overall size of the individual particles. FIGS. 2A to 2B are scanning electron microscopy (SEM) images of silica-coated magnetic nanoparticle according to the present technology. FIG. 2C shows laser diffraction data for silica-coated magnetic nanoparticles according to the present technology.

Use of the current technique, which includes sonication during the synthesis, aids in dispersing the nanoparticle cores thereby allowing uniform silica coating to occur and prevents premature particles aggregation. Estimates by SEM show the final silica-coated Fe₃O₄ nanoparticles are between 10 and 15 nm in diameter. This suggests the core/coating structure is composed of an internal Fe₃O₄ nanoparticle which is approximately 7-10 nm in diameter which incorporates an external uniform shell of silica approximately 1.5 nm in thickness. These sizes have been confirmed using high resolution TEM.

Examples 1-1, 1-2, and 1-3 were prepared using the technique described above, with differing amounts of the TEOS solution. The silica-coated Fe₃O₄ nanoparticles were analyzed by IR and TEM, and they were found to have highly desirable size characteristics and SiO:FeO ratios. FIGS. 2D and 2E are TEM images for Example 1-1, FIGS. 2F and 2G are TEM images for Example 1-2, and FIGS. 2H and 2I are TEM images for Example 1-3. The following table provides the average shell thickness and average core particle size for each of these examples.

| | | | Coating | Core particle |
|---|---|---|---|---|
| | TEOS ml | | thickness | size |
| Example # | (Eq. ml) | SiO: FeO | (nm) | (nm) |
| 1-1 | 11.3 (1.5) | 3.4 | 1.5 ± 0.3 | 11.3 ± 3 |
| 1-2 | 7.5 (1.0) | 2.4 | 1.4 ± 0.3 | 10.6 ± 2.1 |
| 1-3 | 15.0 (2.0) | 4.3 | 2.3 ± 0.6 | 12.6 ± 3.1 |

In the foregoing table, the amount of TEOS used for the reaction is provided in volumes and in equivalents. The ratio of SiO to FeO in the resulting silica-coated nanoparticles was determined by IR analysis. The shell thickness and the core particle size were determined based on the TEM analysis.

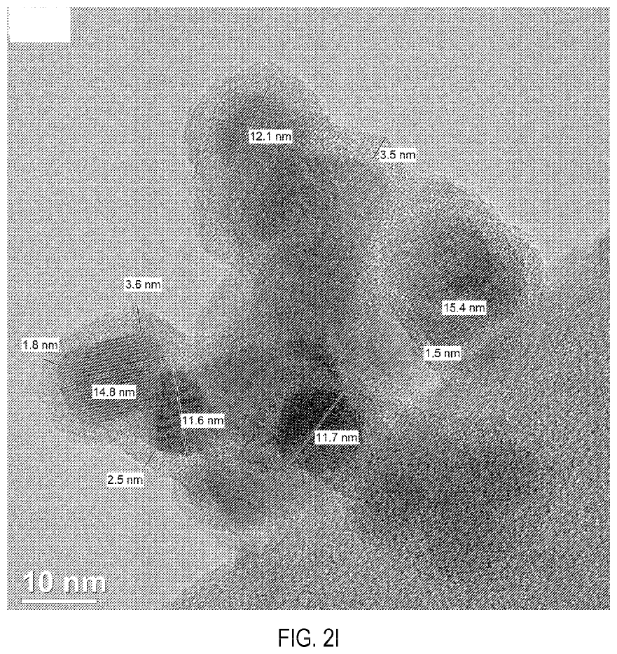

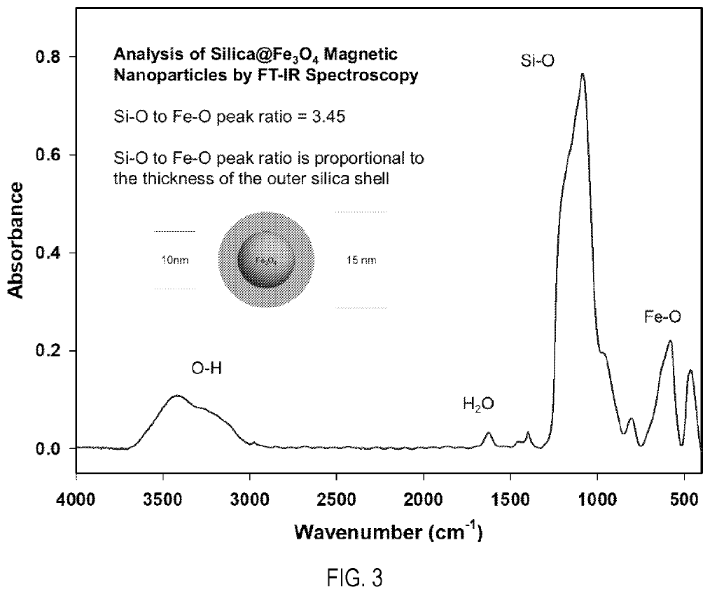

Analysis of the dried silica-coated Fe₃O₄ nanoparticles using Brunauer-Emmett-Teller (BET) theory indicate the materials surface area exceeds 105 m²/g, such as 113-127 m²/g, which matches well with the estimated diameter of the particles. Analysis of the nanoparticles was also performed using FT-IR spectroscopy before and after functionalization with the silica shell. FT-IR analysis of the non-coated Fe₃O₄ nanoparticles shows two overlapping bands at 580 cm⁻¹ and 630 cm⁻¹ which are associated with the metal-oxygen Fe—O stretching vibration in the crystalline lattice of the Fe₃O₄ magnetic phase. After coating the nanoparticles with silica, these bands are joined by additional modes at 3400 cm⁻¹, 1094 cm⁻¹, and 467 cm⁻¹ which arise from silica O—H stretching and Si—O stretching of the SiO₂ shell (See FIG. 3). The appearance of the new modes confirms encapsulation of the nanoparticles with a thin shell of silica.

Two other batches of silica-coated nanoparticles were prepared in the same manner as Example 1-1, and the coating thickness was assessed. As shown in the following table, the procedure resulted in silica-coated nanoparticles having extremely consistent coating thicknesses, with little batch-to-batch variability.

| | SiO₂ Coating |
|---|---|
| Example # | thickness (nm) |
| 1-1 | 1.5 |
| 1-1A | 1.54 |
| 1-1B | 1.56 |

### Example 2

In this example, silica-coated nanoparticles were prepared following the procedure set forth in Example 1, except that different amounts of silicate were used in the procedure. The following tables summarize the five different embodiments of magnetic nanoparticles prepared in this Example.

| Example # | Fe(III)Cl₃ | Fe(II)Cl₂ | TEOS | SiO2@Fe3O4 | IR Ratio |
|---|---|---|---|---|---|
| 2-1 | 2.50 g | 0.93 g | 0.5 ml | 1.25 g | 1.09 |
| 2-2 | 2.50 g | 0.92 g | 1.0 ml | 1.33 g | 1.94 |
| 2-3 | 2.50 g | 0.94 g | 1.5 ml | 1.41 g | 2.84 |
| 2-4 | 2.51 g | 0.95 g | 2.0 ml | 1.63 g | 3.45 |
| 2-5 | 2.50 g | 0.96 g | 2.5 ml | 1.62 g | 4.23 |

| | Wt. SiO₂ | | SiO₂: Fe₃O₄ | SiO₂: Fe₃O₄ | Mass % |
|---|---|---|---|---|---|
| Example # | added | Mols SiO₂ | Molar Ratio | Mass Ratio | Silica |
| 2-1 | 0.18 g | .00300 | .65 to 1 | .17 to 1 | 14.4% |
| | | | (low) | (low) | |
| 2-2 | 0.26 g | .00433 | .94 to 1 | .24 to 1 | 19.5% |
| 2-3 | 0.34 g | .00566 | 1.22 to 1 | .32 to 1 | 24.1% |
| 2-4 | 0.56 g | .00932 | 2.02 to 1 | .52 to 1 | 34.4% |
| | | | (high) | (high) | |
| 2-5 | 0.55 g | .00915 | 1.98 to 1 | .51 to 1 | 34.0% |

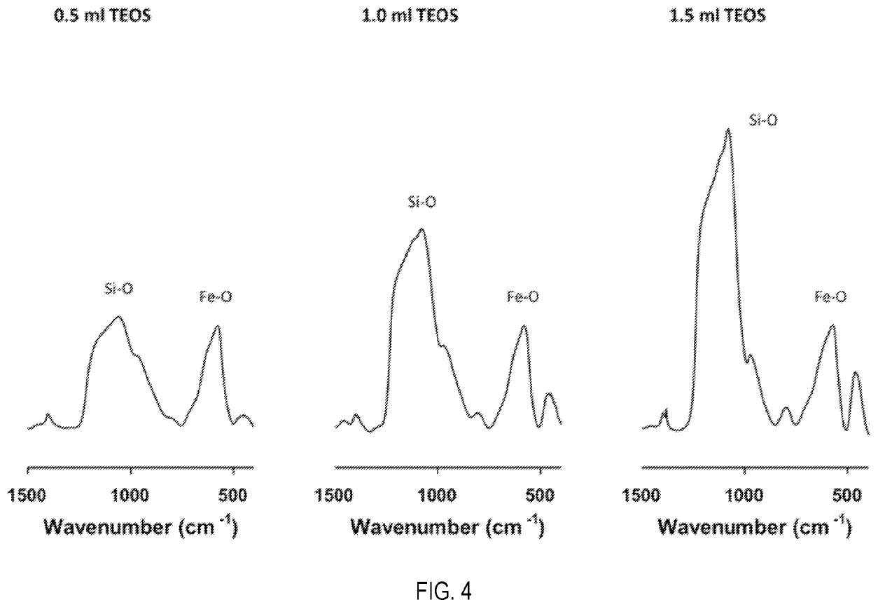

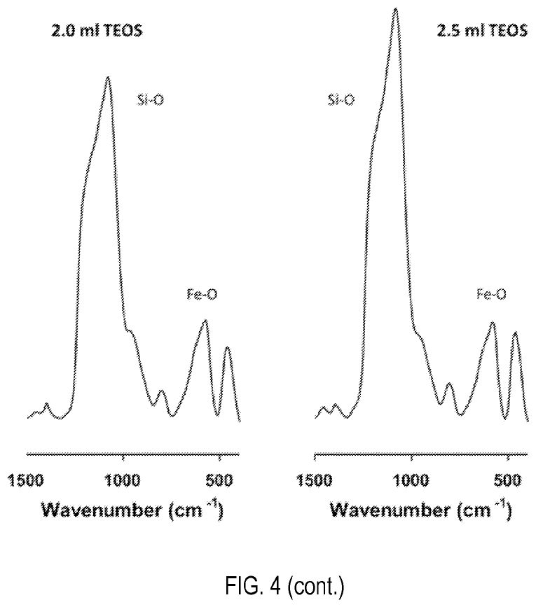

The silica-coated magnetic nanoparticles were analyzed by FT-IR, and FIG. 4 shows the spectra. The particles of the various embodiments have different thicknesses of their silica coatings. The IR analysis demonstrated that the thickness of the silica coating increases linearly with increasing TEOS amounts.

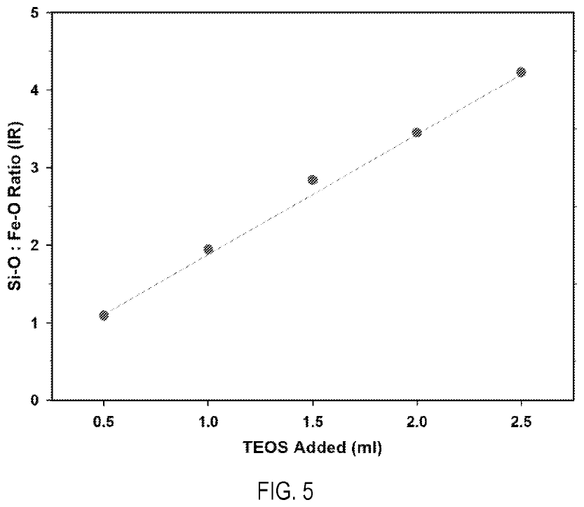

FIG. 5 is a graph illustrating that the thickness of the silica coating increases linearly with increasing TEOS amounts.

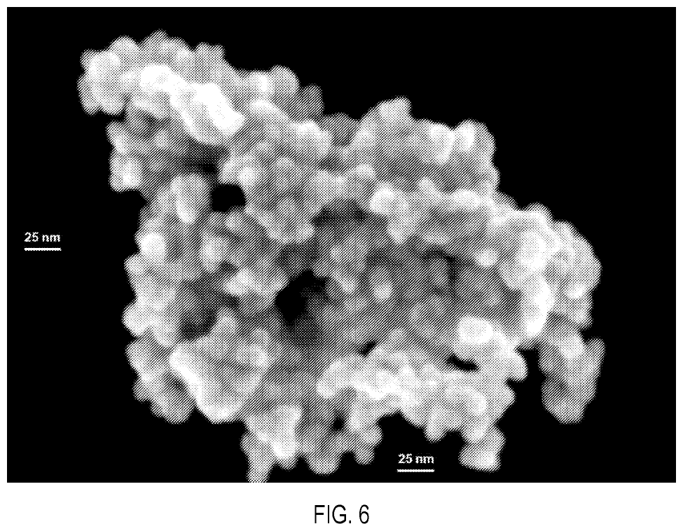

FIG. 6 is a scanning electron microscopy (SEM) image of silica-coated magnetic nanoparticle according to the present technology, at a magnification of 250,000×. The particles were made with 1.5 ml TEOS and sonication.

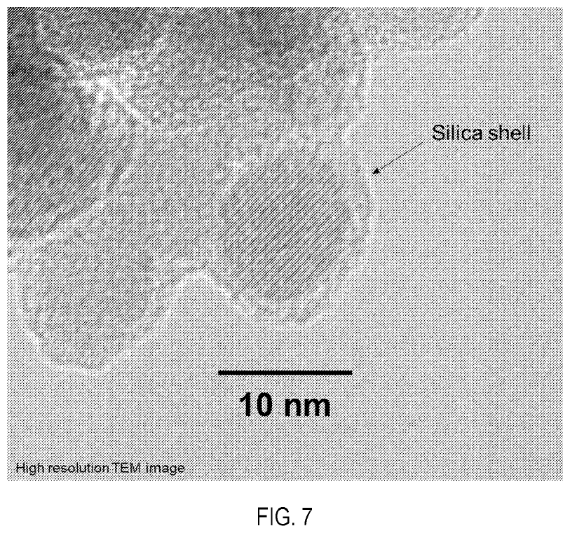

FIG. 7 is a transmission electron microscopy (TEM) image of silica-coated magnetic nanoparticle according to the present technology. Individual silica-coated magnetic nanoparticles are visible. Aggregation of particles is also visible. Typical Fe₃O₄ cores range from 7 to 10 nm. The average silica coating thickness on the particles using 1.5 ml of TEOS was about 1.6 nm. A Fe₃O₄ crystalline lattice is visible inside core, and this indicates the nanoparticles were not oxidized during synthesis or shipping.

### Example 3

In this example, several lots of silica-coated magnetic nanoparticles were prepared following the procedure set forth in Example 1, thereby producing Examples 3-1 to 3-14. The surface areas of several of those examples were determined by Brunauer-Emmett-Teller (BET) surface area, and were found to have surface areas between about 113 m²/g and about 127 m²/g.

| Example # | Surface Area (m²/g) |
|---|---|
| 3-1 | 113 |
| 3-2 | 116 |
| 3-3 | 127 |
| 3-4 | 120 |
| 3-5 | 122 |
| 3-6 | 122 |
| 3-7 | 125 |
| 3-8 | 116 |
| 3-9 | |
| 3-10 | 120 |
| 3-11 | 113 |

### Example 4

In this example, RNA was recovered from a sample using magnetic nanoparticles of the present disclosure in both manual and automated protocols. In these methods, 75 μL aliquots of a biological sample containing RNA were pipetted into 0.2 ml strip tubes. 75 μL guanidine thiocyanate (GTC) containing Lysis Buffer and 2 μL of Proteinase K (20 mg/ml) were added to each tube. The tubes were capped, mixed by vortexing, and spun. The tubes were incubated at 60° C. for 5 minutes. After incubation, 75 μL of 100% EtOH were added to each tube, along with 2 μL of magnetic nanoparticles having a particle size of 10.38 nm, based on a core particle size of 8.82 nm and a coating thickness of 1.56 nm. The tubes were capped, mixed, spun briefly, and incubated for 5 minutes at room temperature. During this second incubation period, RNA from the sample bound to the silica-coated magnetic nanoparticles.

After the second incubation period, the tubes were placed on a magnet for 5 minutes. Then, all supernatants were removed from the tubes without disturbing the nanoparticle pellets. 200 μL of washing medium (80% EtOH) was added to the pellets in the tubes. The wash medium was removed without disturbing the nanoparticle pellet and the wash and removal steps were repeated. The nanoparticle pellet was incubated at room temperature for 3 minutes to dry the nanoparticles.

75 μL of elution medium pre-heated to 65° C. was then added to the tubes and it pipetted up and down several times to mix the nanoparticles while avoiding production of air bubbles. The tubes were allowed to stand for 2 minutes, nanoparticles were captured by a magnet and the eluate containing the RNA was recovered and transferred to a fresh tube. The fresh tube was capped and placed on ice.

### Example 5

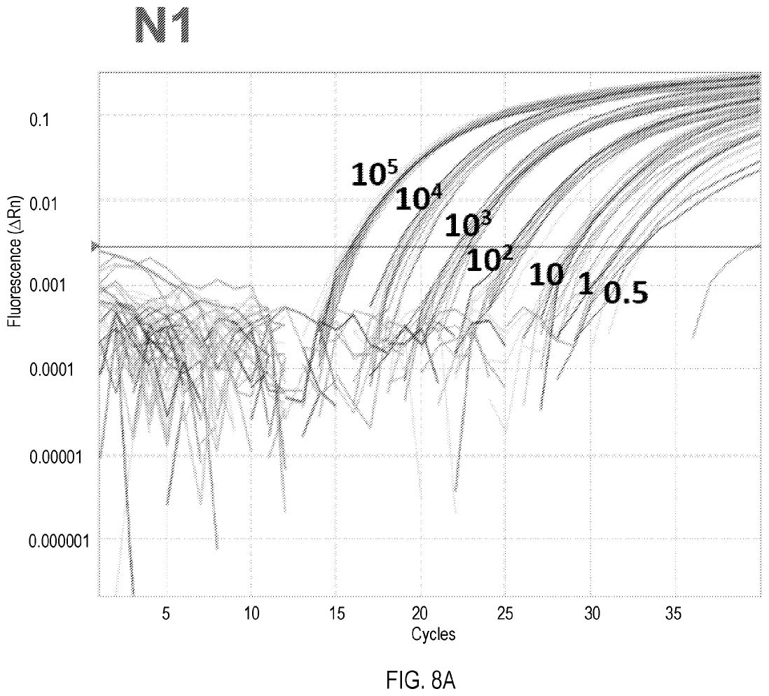

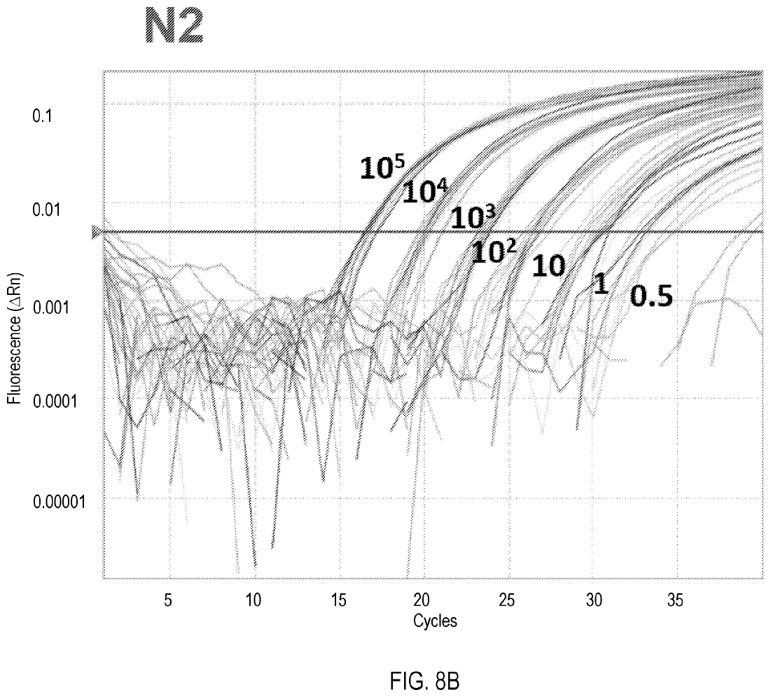

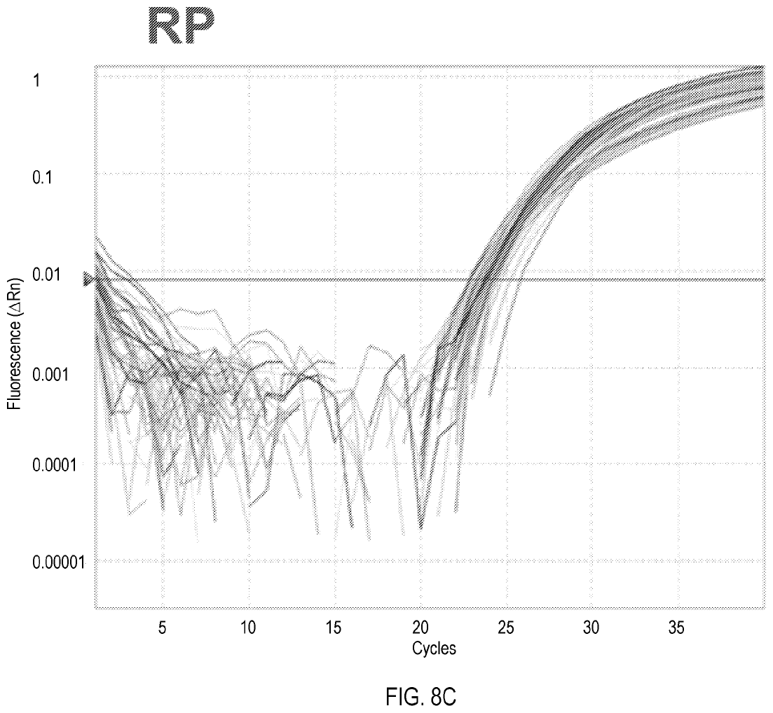

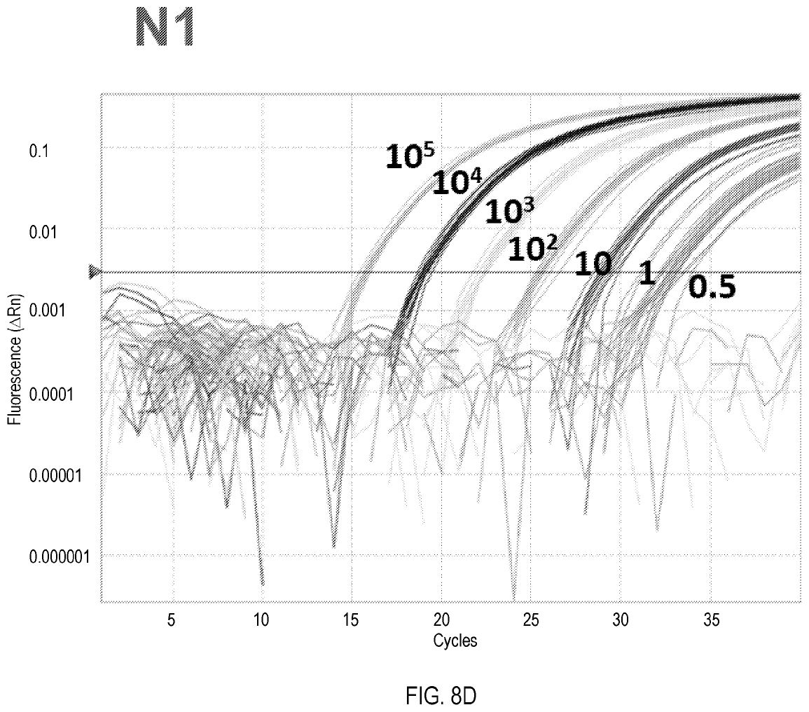

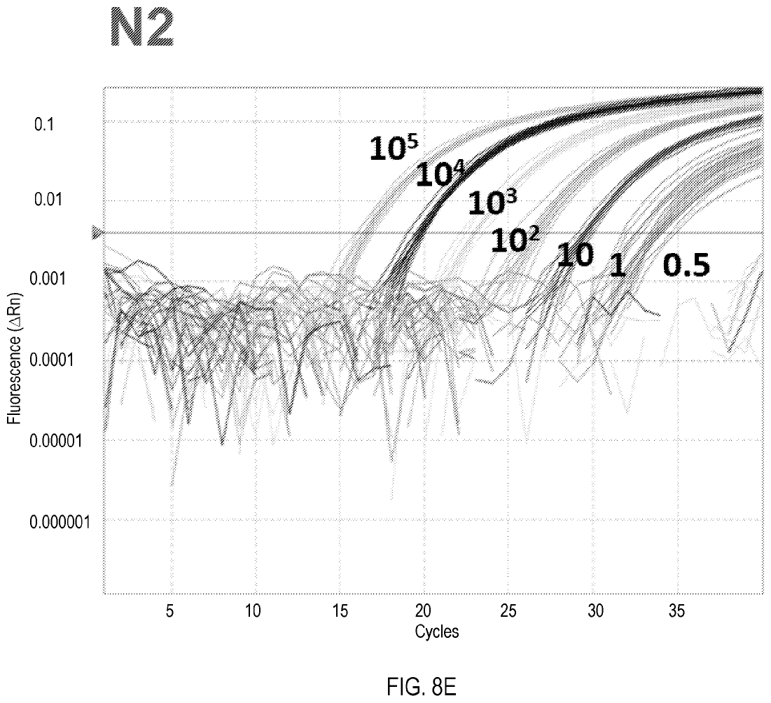

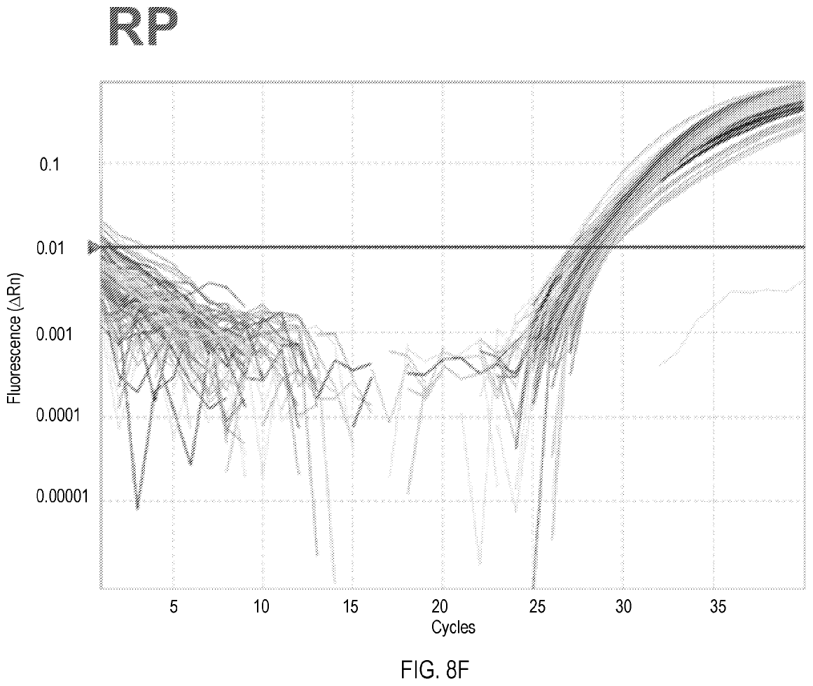

In this example, RNA was recovered from a sample using magnetic nanoparticles of the present disclosure in a manual protocol. FIG. 8A provides a comparison of six nanoparticles (NP) lots for viral RNA extraction using the manual workflow.

Contrived samples were prepared by spiking-in SARS-COV-2 armored synthetic RNA into Nasal Swab (NS) collected into VTM to the final concentrations of SARS-COV-2 N1 and N2 targets 105, 104, 103, 102, 10, 1 and 0.5 copies per microliter. RNA extraction was performed using 75 ul of each dilution in duplicate following the NAP manual protocol. Shortly, 75 ul of each sample was mixed with 75 ul of the lysis buffer and 2 ul of Proteinase K, incubated at RT for 5 min and at 60° C. for 5 min, then 75 ul of 100% ethanol containing 2 ul of nanoparticle beads were added. The following 6 nanoparticle lots were used in this experiment: Example 3-3, Example 3-5, Example 3-6, Example 3-7, Example 3-8, and Example 3-9. Samples were mixed and incubated at RT for 5 min to allow nucleic acids bind to magnetic beads, then tubes were transferred to a magnet stand and beads were collected at the bottom side of the tubes (RNA remains bound to magnetic beads at this step). After aspirating the supernatant, beads were washed twice with 80% ethanol, dried for 3 min. at RT and RNA was eluted with 30 ul of Low TE buffer (10 mM Tris, 0.1 mM EDTA, pH 8.0).

5 ul of each extracted RNA sample were used in multiplex QRT-PCR reactions with primers and probes specific to SARS-COV-2 N1 and N2 targets and human RNase P gene. The assay was run on the Agilent AriaMx Real-Time PCR system. Amplification curves of N1, N2 and RP targets presented in FIGS. 8A, 8B and 8C and corresponding Cq's presented in Tables A, B and C. No significant difference was observed between six nanoparticle lots and QRT-PCR detection of SARS-COV2 N1 and N2 RNA targets at 10⁵-0.5 copies/ul and human RP were demonstrated.

TABLE A

| SARS | N1 Cq (ΔRn) | | | | | |
|---|---|---|---|---|---|---|
| (copies/uL) | Ex. 3-3 | Ex. 3-5 | Ex. 3-6 | Ex. 3-7 | Ex. 3-8 | Ex. 3-9 |
| 100,000.00 | 16.54 | 16.31 | 16.16 | 16.3 | 16.39 | 16.23 |
| 10,000.00 | 19.74 | 20.15 | 19.25 | 19.47 | 19.3 | 19.29 |
| 1,000.00 | 22.96 | 22.9 | 22.34 | 22.57 | 22.71 | 22.77 |
| 100 | 27.59 | 26.49 | 25.77 | 25.79 | 26.11 | 25.94 |
| 10 | 30.29 | 30.14 | 29.18 | 29.21 | 29.51 | 30.03 |
| 1 | 33.09 | 31.63 | 32.2 | 32.19 | 33.52 | 32.25 |
| 0.5 | 33.29 | 32.24 | 31.46 | 33.54 | 32.85 | 31.62 |
| 0 | No Ct | No Ct | No Ct | No Ct | No Ct | No Ct |

TABLE B

| SARS | N2 Cq (ΔRn) | | | | | |
|---|---|---|---|---|---|---|
| (copies/uL) | Ex. 3-3 | Ex. 3-5 | Ex. 3-6 | Ex. 3-7 | Ex. 3-8 | Ex. 3-9 |
| 100,000.00 | 16.82 | 16.65 | 16.74 | 16.81 | 16.88 | 17 |
| 10,000.00 | 20.49 | 20.56 | 19.9 | 20.4 | 20.13 | 19.83 |
| 1,000.00 | 23.6 | 23.69 | 23.54 | 23.46 | 23.4 | 23.76 |
| 100 | 27.79 | 27.19 | 26.7 | 26.69 | 26.86 | 26.68 |
| 10 | 31.25 | 30.79 | 30.18 | 29.76 | 30.64 | 30.87 |
| 1 | 35.45 | 33.14 | 34.19 | 33.49 | 34.8 | 34.69 |
| 0.5 | 35.33 | 33.69 | 33.58 | 37.61 | 36.15 | 33.2 |
| 0 | No Ct | No Ct | No Ct | No Ct | No Ct | No Ct |

TABLE C

| SARS | RP Cq (ΔRn) | | | | | |
|---|---|---|---|---|---|---|
| (copies/uL) | Ex. 3-3 | Ex. 3-5 | Ex. 3-6 | Ex. 3-7 | Ex. 3-8 | Ex. 3-9 |
| 100,000.00 | 23.14 | 22.6 | 22.91 | 22.91 | 23.34 | 23.18 |
| 10,000.00 | 23 | 23.38 | 22.95 | 23.22 | 23.12 | 22.96 |
| 1,000.00 | 23.71 | 23.77 | 23.61 | 23.56 | 23.69 | 23.83 |
| 100 | 25.49 | 24.72 | 23.92 | 23.64 | 24.14 | 24.05 |
| 10 | 24.23 | 24.51 | 24.36 | 24.13 | 23.86 | 23.78 |
| 1 | 23.63 | 24.25 | 24.15 | 23.96 | 23.85 | 23.47 |
| 0.5 | 24.32 | 24.19 | 23.95 | 24.37 | 24.17 | 23.83 |
| 0 | 24.37 | 23.77 | 23.88 | 23.84 | 24.55 | 23.66 |

### Example 6

In the following example, RNA was recovered from a sample using magnetic nanoparticles of the present disclosure in an automated protocol. FIGS. 8D, 8E and 8F show a comparison of six nanoparticles lots for viral RNA extraction using an automation workflow.

Contrived samples were prepared by spiking-in SARS-COV-2 armored synthetic RNA into Nasal Swab (NS) collected into viral transfer medium (VTM) to the final concentrations of SARS-COV-2 N1 and N2 targets 10⁵, 10⁴, 10³, 10², 10, 1 and 0.5 copies per microliter. RNA extraction was performed using 75 ul of each dilution. Shortly, 75 ul of each sample was mixed with 75 ul of the lysis buffer and 2 ul of Proteinase K, incubated at RT for 5 min and then transferred into the Bravo instrument for an automatic processing. The following nanoparticle lots were used in this experiment: Example 3-2, Example 3-3 (from two different vials, designated 3-3a and 3-3b), Example 3-4, Example 3-5, and Example 1-1B. RNA was eluted with 30 ul of Low TE buffer (10 mM Tris, 0.1 mM EDTA, pH 8.0).

5 ul of each extracted RNA sample were used in multiplex QRT-PCR reaction with primers and probes specific to SARS-COV-2 N1 and N2 targets and human RNase P gene. The assay was run on the Agilent AriaMx Real-Time PCR system. Amplification curves of N1, N2 and RP targets presented in FIGS. 8D, 8E and 8F and corresponding Cq's presented in Tables D, E and F. No significant differences were observed between six nanoparticle lots and QRT-PCR detection of SARS-COV2 N1 and N2 RNA targets at 10⁵-0.5 copies/ul and human RP were demonstrated.

TABLE D

| | N1 Cq (ΔRn) | | | | | |
|---|---|---|---|---|---|---|
| SARS | Ex. | Ex. | Ex. | Ex. | Ex. | Ex. |
| (copies/uL) | 3-2 | 3-3a | 3-4 | 3-5 | 1-1B | 3-3b |
| 100,000.00 | 15.88 | 17.79 | 15.92 | 15.68 | 15.66 | 15.69 |
| 10,000.00 | 19.22 | 19.32 | 19.00 | 18.95 | 18.83 | 19.02 |
| 1,000.00 | 22.42 | 22.54 | 22.30 | 23.01 | 21.93 | 22.04 |
| 100.00 | 25.68 | 25.83 | 26.60 | 25.96 | 25.67 | 25.40 |
| 10.00 | 29.02 | 29.41 | 29.12 | 28.68 | 29.53 | 29.14 |
| 1.00 | 32.85 | 31.85 | 31.80 | 32.65 | 32.94 | 32.76 |
| 0.5 | No Ct | 33.36 | 34.03 | 33.18 | 33.37 | 32.72 |
| 0 | No Ct | No Ct | No Ct | No Ct | No Ct | No Ct |

TABLE E

| | N2 Cq (ΔRn) | | | | | |
|---|---|---|---|---|---|---|
| SARS | Ex. | Ex. | Ex. | Ex. | Ex. | Ex. |
| (copies/uL) | 3-2 | 3-3a | 3-4 | 3-5 | 1-1B | 3-3b |
| 100,000.00 | 16.60 | 27.46 | 16.89 | 16.74 | 16.42 | 16.28 |
| 10,000.00 | 19.84 | 19.91 | 19.90 | 19.84 | 19.59 | 19.81 |
| 1,000.00 | 23.19 | 23.43 | 23.13 | 23.79 | 22.88 | 22.64 |
| 100.00 | 27.03 | 27.02 | 27.93 | 26.87 | 26.59 | 26.07 |
| 10.00 | 29.67 | 29.72 | 29.63 | 29.44 | 29.92 | 29.80 |
| 1.00 | 33.40 | 33.22 | 32.97 | 32.45 | 34.28 | 33.77 |
| 0.5 | 34.76 | 34.17 | 33.95 | 34.12 | 34.08 | 33.31 |
| 0 | No Ct | No Ct | No Ct | No Ct | No Ct | No Ct |

TABLE F

| | RP Cq (ΔRn) | | | | | |
|---|---|---|---|---|---|---|
| SARS | Ex. | Ex. | Ex. | Ex. | Ex. | Ex. |
| (copies/uL) | 3-2 | 3-3a | 3-4 | 3-5 | 1-1B | 3-3b |
| 100,000.00 | 28.72 | 28.78 | 28.97 | 28.89 | 28.96 | 29.31 |
| 10,000.00 | 27.77 | 28.03 | 27.93 | 27.82 | 28.00 | 27.60 |
| 1,000.00 | 27.96 | 27.52 | 27.85 | 27.98 | 27.65 | 27.50 |
| 100.00 | 28.44 | 28.63 | 27.93 | 28.54 | 28.44 | 28.14 |
| 10.00 | 28.51 | 28.96 | 28.68 | 28.47 | 28.44 | 29.03 |
| 1.00 | 28.88 | 28.52 | 28.91 | 28.71 | 29.06 | 28.71 |
| 0.5 | 29.01 | 29.01 | 29.20 | 29.02 | 28.98 | 28.98 |
| 0 | 28.13 | 28.72 | 28.78 | 29.28 | 28.85 | 29.32 |

### Example 7

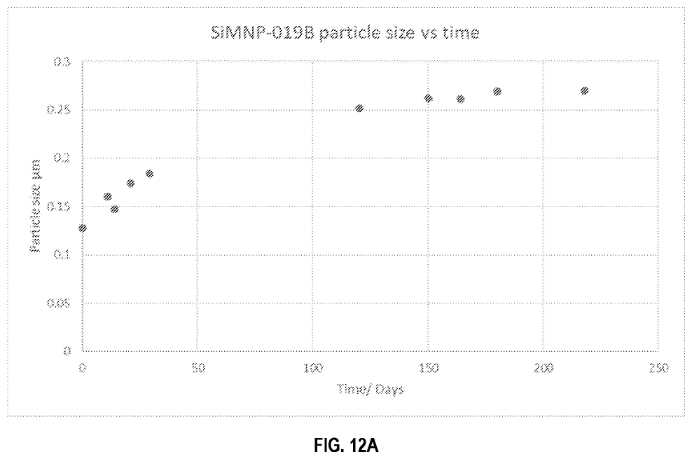

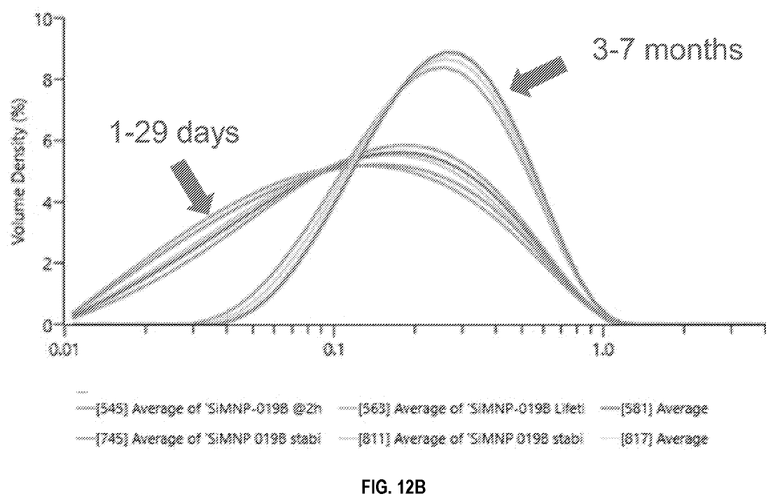

In this experiment, the NP aging effect on average cluster size was evaluated. FIGS. 12A and 12B illustrate the increase in cluster size as a nanoparticle composition of the present disclosure was aged up to 218 days. Table H shows values measured at the indicated time points.

TABLE H

| Mode (μm) | Time (days) |
|---|---|
| 0.128 | 0 |
| 0.16 | 11 |
| 0.147 | 14 |
| 0.174 | 21 |
| 0.184 | 29 |
| 0.252 | 120 |
| 0.262 | 150 |
| 0.261 | 164 |
| 0.269 | 180 |
| 0.27 | 218 |

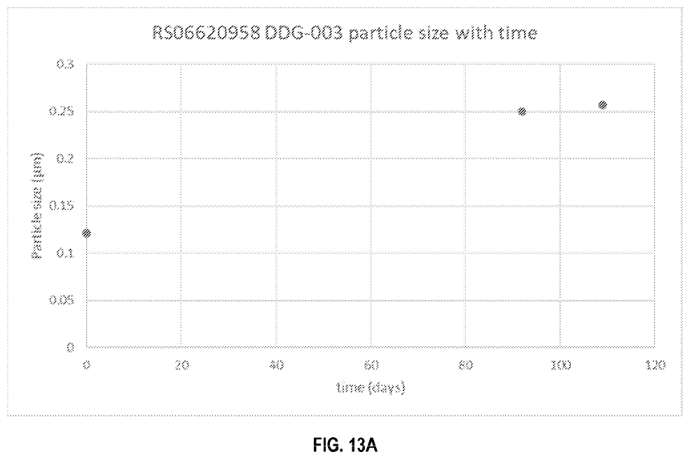

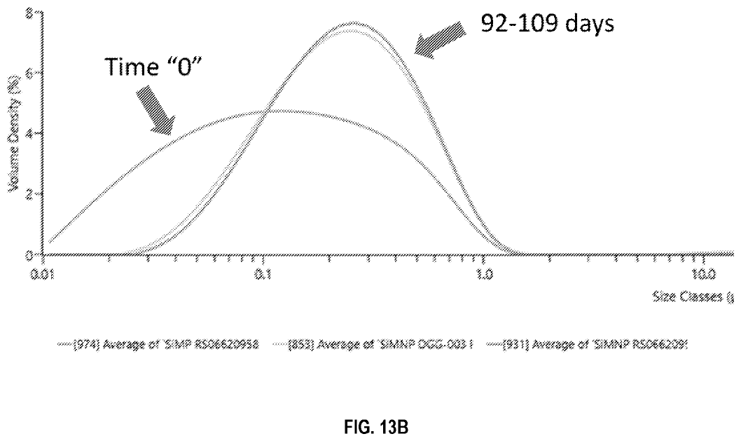

FIGS. 13A and 13B illustrate another experiment which demonstrates the increase in cluster size as another nanoparticle composition of the present disclosure was aged up to 109 days. Table I shows values measured at the indicated time points.

TABLE I

| Mode (μm) | Time (days) |
|---|---|
| 0.121 | 0 |
| 0.25 | 92 |
| 0.257 | 109 |

These experiments show that nanoparticle mode size increased over a cluster period and leveled off at approximately 300 nm, more particularly about 260 nm. Nanoparticle compositions comprising clusters of this size were used for the exemplary RNA isolation/QRT-PCR assays and NGS normalization.

### Example 8

In this example, the NP aging effect on nucleic acid extraction efficiency was evaluated by comparing QRT-PCR analysis of RNA isolated using NP that were 2 and 14 weeks old. Nucleic acids were extracted from 14 Nasopharyngeal (NP) clinical samples, 8 of which were known positive for human metapneumovirus (hMPV) and 6 positive for human parainfluenza 3 virus (hPIV3). A quantity of extracted viral RNA was evaluated by QRT-PCR on the AriaMx Real-Time PCR System using Brilliant III QRT-PCR reagents, ABI TaqMan microbial assays (primers and probes to detect hMPV or hPIV3) and 2 ul of each RNA sample in 20 μl PCR reaction. The results were analyzed using the AriaMx v1.5 software (ΔCq values for each sample were calculated as [ΔCq=Cq 2-weeks-Cq 14 weeks]) and the data is shown the table below.

As shown in Table G, ten out of fourteen samples (71%) extracted using 14 week old NP resulted in >0.5 Cq Improvement (ΔCq 1-3 fold difference in RNA yield).

TABLE G

| NP swabs | | | | |
|---|---|---|---|---|
| Samples | | QPCR Cq | | Delta Cq [DCq] |
| # | ID | 2 weeks | 14 weeks | Cq 2 weeks − Cq 14 weeks |
| 1 | MPV1 | 27.4 | 25.3 | 2.1 |
| 2 | MPV2 | 32.6 | 32.1 | 0.5 |
| 3 | MPV7 | 30.5 | 28.6 | 1.9 |
| 4 | MPV13 | 23.7 | 22.5 | 1.2 |
| 5 | MPV15 | 31.3 | 29.4 | 1.9 |
| 6 | MPV16 | 30.8 | 30 | 0.8 |
| 7 | MPV17 | 28.5 | 27 | 1.5 |
| 8 | MPV18 | 29.3 | 28 | 1.3 |
| 9 | PIV3-2 | 23.9 | 23.1 | 0.8 |
| 10 | PIV3-3 | 18.9 | 17.4 | 1.5 |
| 11 | PIV3-4 | 24.4 | 24.1 | 0.3 |
| 12 | PIV3-9 | 23.8 | 23.4 | 0.4 |
| 13 | PIV3-10 | 32.4 | 30.3 | 2.1 |
| 14 | PIV3-11 | 19.8 | 19.7 | 0.1 |

This experiment demonstrated that a fourteen week old NP composition (an “aged” NP composition) resulted in significant improvement in viral RNA yield from clinical samples as measured by QRT-PCR.

### Example 9

In the following example, DNA samples destined for NGS, referred to as “libraries”, having different concentrations were “normalized”, meaning that regardless of the DNA concentration to which magnetic silica nanoparticles were exposed, all resulting DNA concentrations following normalization possessed approximately equal concentrations.

The following steps were all performed at 4° C.

Add 10 μL (0.1 μg/mL; 1 μg) of magnetic nanoparticles and 60 μL of DNA binding medium into tubes A1-H1 (nanoparticles lot CS-019A) and A2-H2 (nanoparticles CS-019B) and mix by pipetting.

Add 20 μL of 10 nM DNA libraries into tubes A1-D1 and A2-D2 and 20 μL of 100 nM DNA libraries into tubes E1-H1 and E2-H2, mix by pipetting and immediately capture the particles.

Wash the captured particles once with 150 μL of 80% EtOH, remove supernatants, dry particles, add 10 μL of 10 mM Tris-0.1 mM EDTA (pH 8.0), mix, immediately capture particles and perform electrophoretic analysis (HSD1000 TapeStation) on the supernatants.

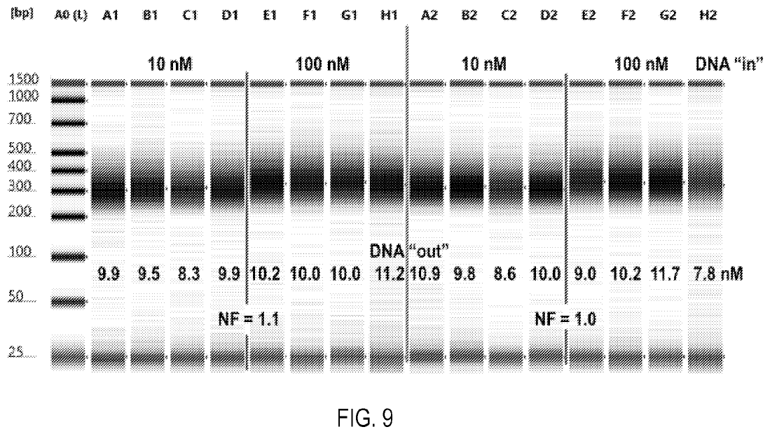

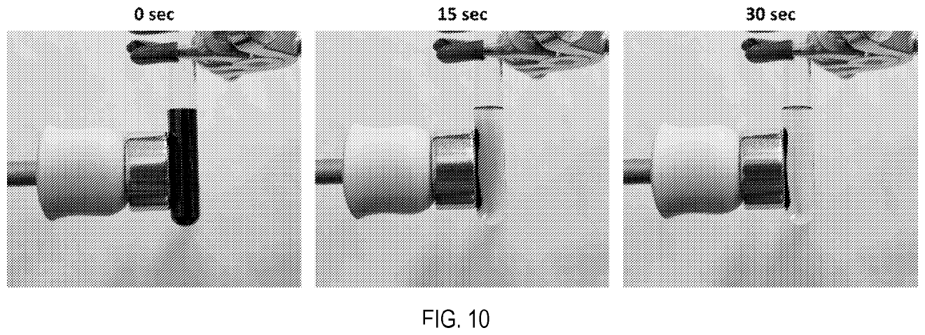

FIG. 9 shows the results of TapeStation analysis. Despite different concentrations of nucleic acid in the DNA libraries used as inputs for the normalization method, Normalization Factors were 1.1 for CS-019A and 1.0 for CS-019B. Average DNA concentrations from tubes A1 to D1 and E1-H1 (CS-019A) were 9.4 nM and 10.4 nM, respectively. 10.4 divided by 9.4 results in a normalization factor of essentially 1.1. Average DNA concentrations from tubes A2 to D2 and E2 to H2 (CS-019B) were 9.8 nM and 9.7 nM, respectively. 9.8 divided by 9.7 results in a normalization factor of essentially 1.0.

### Example 10

In this example, DNA samples (or libraries) for NGS having different concentrations were normalized. This example further demonstrates that NGS protocols can be improved by using NP to normalize variable concentration nucleic acid libraries resulting in higher sample throughput and reduced sequencing instrument time and costs. NGS instrument platforms generally require a specific concentration of nucleic acids to be loaded onto the sequencing instrument. Concentrations too low or too high results in low sequence data output and quality. There are several existing approaches to normalize variable concentration nucleic acids for NGS. These include nucleic acid quantification by spectrophotometry, fluorimetry, quantitative PCR, or electrophoresis followed by calculation of desired concentrations and then dilution of all samples to a normalized concentration. The following NGS data comparison shows equal or higher quality of NP normalization compared to electrophoresis (Agilent TapeStation device).

The normalization procedure set forth in Example 8 was used with silica-coated magnetic nanoparticles prepared according to Example 1 (nanoparticles lot CS-019).

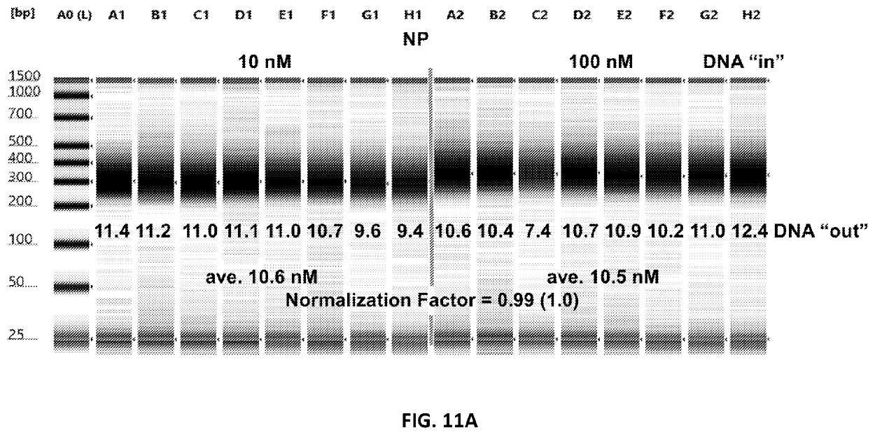

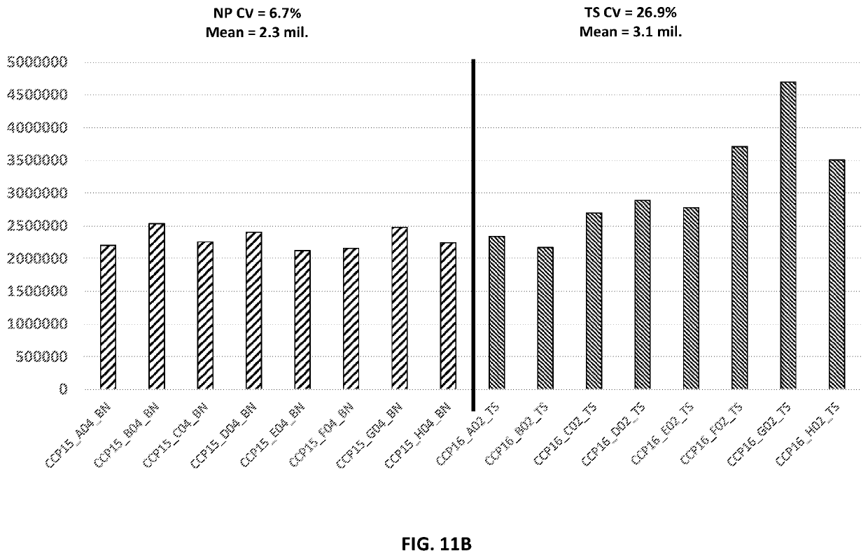

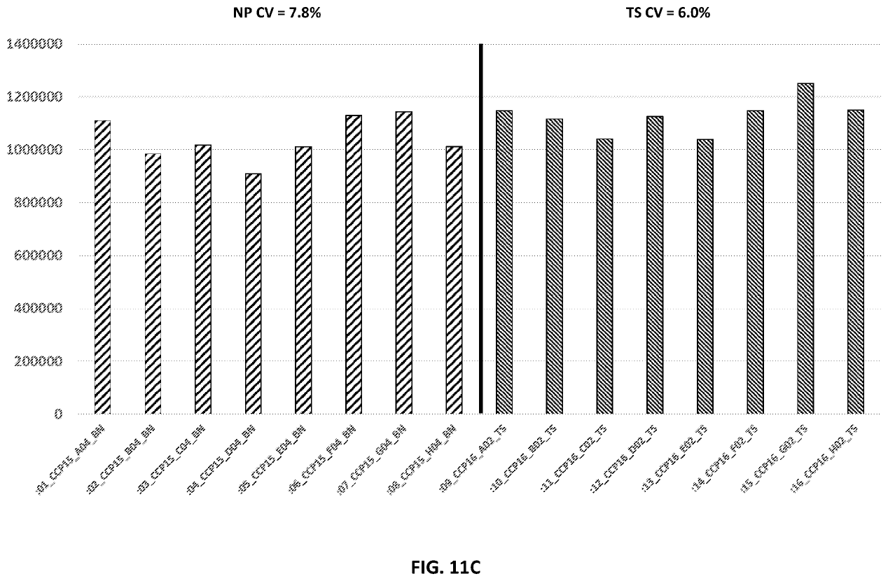

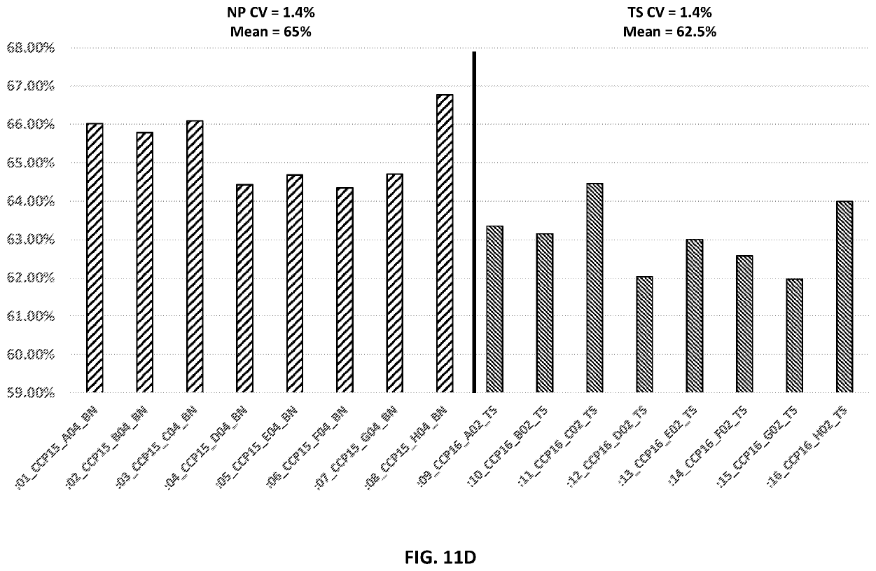

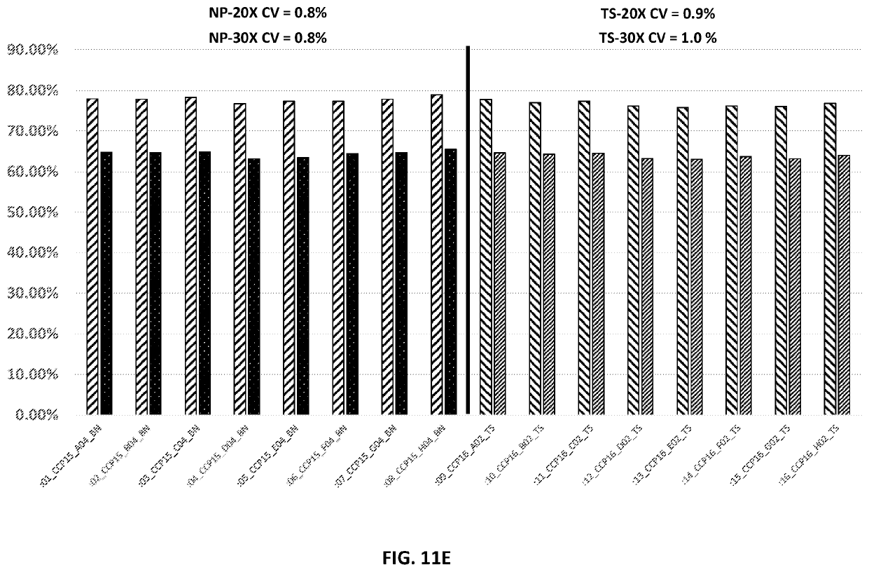

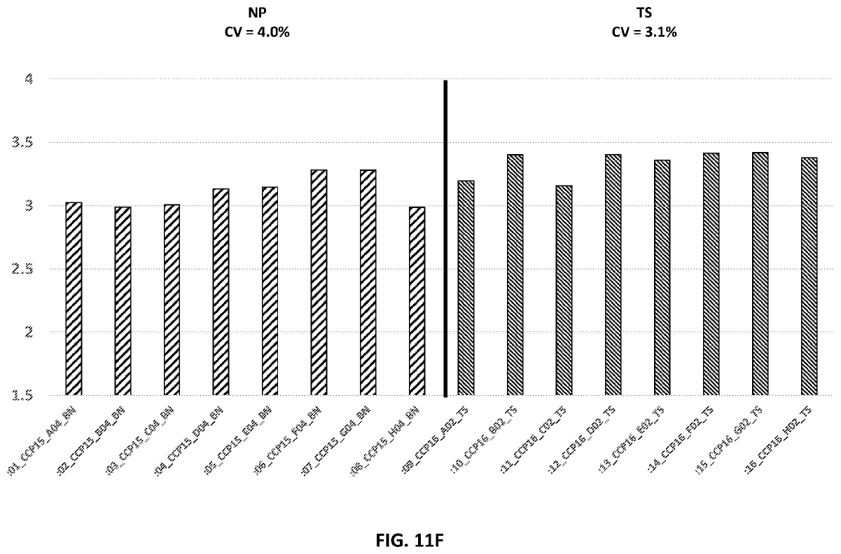

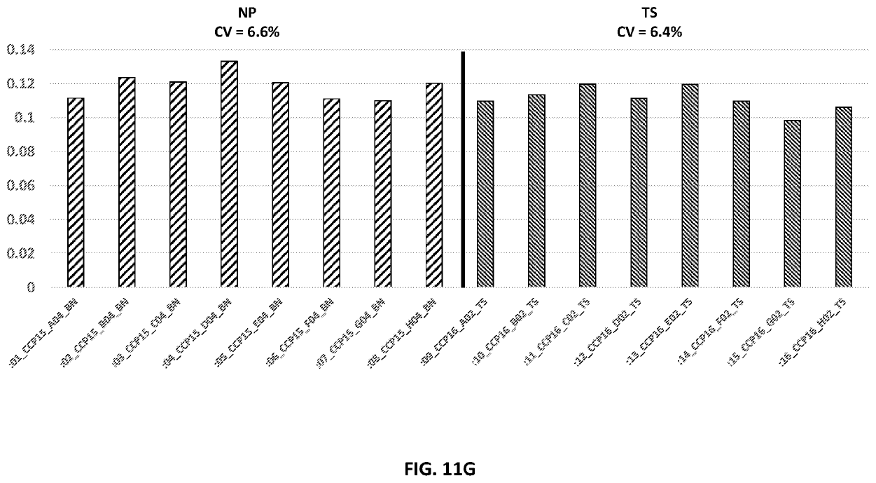

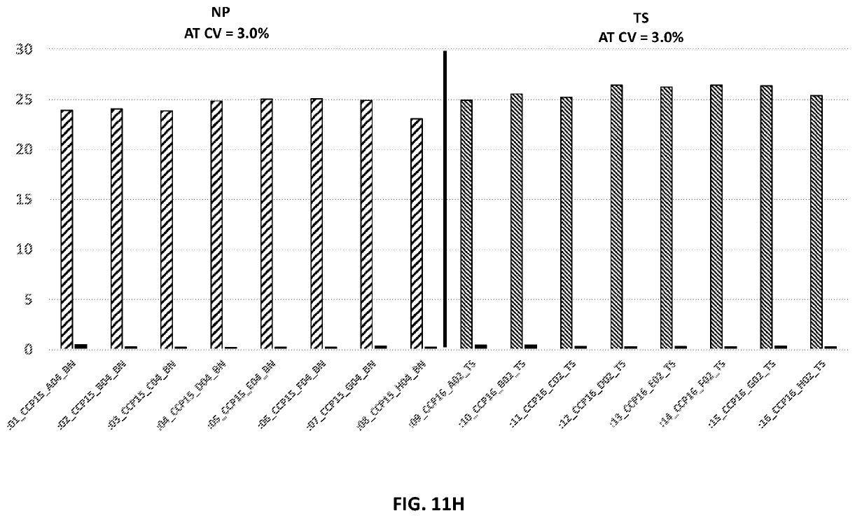

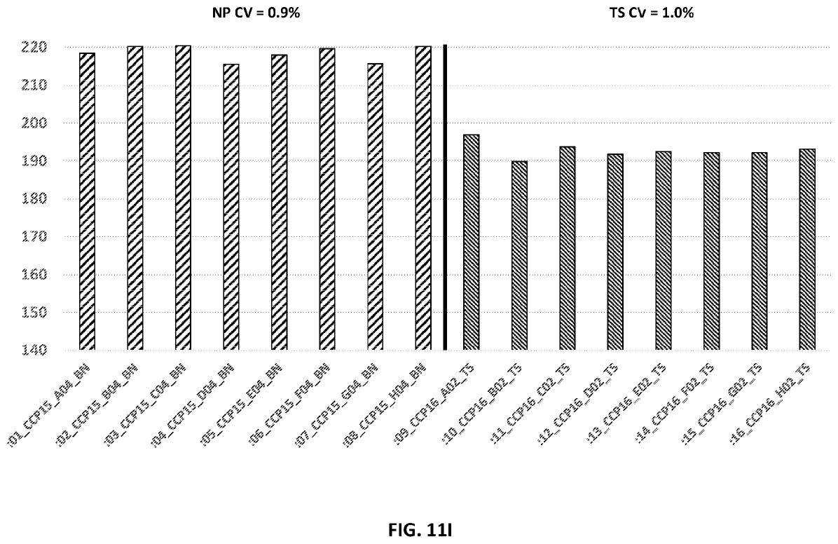

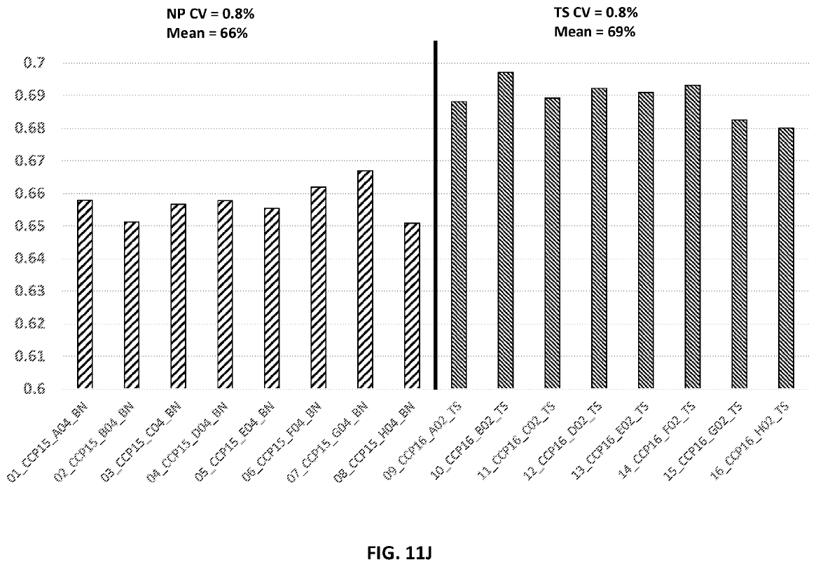

The results are illustrated in FIGS. 11A to 11J. In those figures, BN refers to NP normalized libraries and TS refers to TapeStation normalized libraries. FIG. 11A shows the results of TapeStation analysis.

FIG. 11B shows “bam counts”, which are the number of sequence reads mapped to each feature of interest such as genes, exons, transcripts, etc. The bam counts were lower for NP-normalized libraries compared to TS-normalized libraries, but they were more uniform.

FIG. 11C shows the complexity of the normalized libraries, which reflects the amount of bias created by a given experimental design. In terms of library complexity, the ideal is a highly complex library that reflects with high fidelity the original complexity of the source material. NP and TS normalization produced equal complexity as shown by nearly equal coefficients of variation. The NP-normalized libraries and TS-normalized libraries were equally complex.

FIG. 11D shows the uniformity of the normalized libraries, which is the percentage of targeted base positions in which the read depth is greater than a prescribed number of times the mean region target coverage depth. The NP-normalized libraries were more uniform than the TS-normalized libraries.

FIG. 11E shows coverage of the normalized libraries. The number of times that a particular base within the target region is represented in the sequence data is referred to as coverage or depth of coverage. The number before the ‘x’ is the coverage (the average number of times your genome will be sequenced). For example, when one has 30× whole genome sequencing (WGS), the ‘30×’ means that the entire genome will be sequenced an average of 30 times. At both 20× and 30× coverage, NP and TS normalization provided equal results.

FIG. 11F shows “Fold 80” base pair penalty, which is the fold of additional sequencing required to ensure that 80% of the target bases achieve the mean coverage. The lower the on-target rate, or the higher the fold-80, the higher the capture inefficiency and wasted sequencing. NP normalization resulted in an average “Fold 80” of 3 while TS normalization resulted in an average “Fold 80” of 3.25, meaning less sequencing is required for the NP-normalized libraries compared to TS-normalized libraries. The lower the Fold 80 number, the better the result.

FIG. 11G shows duplications in the normalized libraries. When multiple sequencing reads are mapped to the exact same location, including the coordinates of the 3′ and 5′ ends, they are considered duplicate reads. The duplication rate is the fraction of mapped reads marked as duplicate reads in a particular data set. In contrast to overlapping reads, duplicate reads offer no additional information and are removed from sequencing data during bioinformatic analysis, a process known as deduplication. NP normalization and TS normalization provided equal duplication values.

FIG. 11H shows AT/GC Dropout. The AT/GC dropout metrics indicate the degree of inadequate coverage of a particular region based on its AT or GC content. For the NP-normalized libraries and TS-normalized libraries, the values for these metrics were equal.

FIG. 11I shows Mean Insert Size. Insert size is the length of the DNA (or RNA) to be sequenced and that is “inserted” between the adapters. For the NP-normalized libraries, the mean insert size was 220 bp, and for the TS-normalized libraries, the mean insert size was 190 bp, differing by 30 bp higher for NP normalization.

FIG. 11J shows On-Target values for the normalized libraries. The percent bases on-target is the number of bases that map to the target region, while the percent reads on-target includes all sequencing reads that overlap the target region by even by a single base. The more bases or reads that fall within the target region, the greater the overall on-target rate—indicating strong probe specificity, high-quality probes, and efficient hybridization-based target enrichment. The NP-normalized libraries and TS-normalized libraries differed by only 0.03%.

In view of this disclosure, it is noted that the present methods, compositions, and systems can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

1. A composition comprising magnetic nanoparticles in a liquid medium, the magnetic nanoparticles comprising a magnetic core and a coating on the core, the coating having a thickness from about 1 nm to about 2 nm; and the magnetic nanoparticles in the composition have a Brunauer-Emmett-Teller (BET) surface area of from about 110 m²/g to about 130 m²/g
the composition comprising clusters of individual magnetic nanoparticles having an average cluster size of from about 250 nm to about 350 nm.

2-3. (canceled)

4. The composition of claim 1, wherein the individual magnetic nanoparticles have an average total diameter of about 15 nm or less.

5-6. (canceled)

7. The composition of claim 1, wherein the cores are single domain magnetic nanoparticles of Fe₃O₄ which display paramagnetism.

8. The composition of claim 1, wherein the cores are single nanoscale crystals of magnetite.

9. The composition of claim 1, wherein the cores are substantially free of Fe₂O₃.

10. The composition of claim 1, wherein the coating comprises silica.

11. The composition of claim 1, wherein the coating further comprises a binding moiety that binds an analyte.

12. The composition of claim 11, wherein the analyte is nucleic acid.

13-15. (canceled)

16. The composition of claim 15, wherein the clusters have an average cluster size of about 300 nm.

17. The composition of claim 1, wherein the composition is a stable suspension of the magnetic nanoparticles, and the magnetic nanoparticles are present in at a concentration of from about 3 to about 30 g/L.

18. The composition of claim 17, wherein the magnetic nanoparticles remain suspended for at least 6 months at a temperature of about 25° C.

19-35. (canceled)

36. A method for preparing a nucleic acid preparation for analysis, the method comprising the steps of:
combining a sample comprising nucleic acids with a binding medium and a magnetic nanoparticle composition in a vessel,
the magnetic particle composition comprising a plurality of magnetic nanoparticles comprise having a core and a coating on the core the coating having a thickness of from about 1.5 nm to about 2 nm, the nucleic acids binding to the coating of the magnetic nanoparticles;
separating the nucleic acid-bound magnetic nanoparticles from liquid within the vessel using a magnet;
removing the liquid from the vessel and retaining the nucleic acid-bound magnetic nanoparticles within the vessel;
washing the nanoparticles using a washing medium to remove contaminants while the nanoparticles are still attracted to the magnet inside the vessel;
removing the washing medium;
contacting the nucleic acid-bound magnetic nanoparticles with an elution medium to separate the bound nucleic acids from the magnetic nanoparticles;
separating the magnetic nanoparticles from nucleic acid containing elution medium within the vessel using a magnet; and
removing the nucleic acids from the vessel.

37. (canceled)

38. The method of claim 36, wherein the washing medium comprises at least 80% v/v alcohol.

39. The method of claim 36, wherein the coating of the magnetic nanoparticles comprises a binding moiety configured to bind the nucleic acid.

40. The method of claim 36, wherein the magnetic nanoparticle composition has a normalization factor between 0.8 and 1.2.

41. (canceled)

42. The method of claim 36, wherein the nucleic acid preparation comprises nucleic acids having lengths within a predetermined length range.

43. The method of claim 36, wherein the sample comprising nucleic acids has less than 200 ng of nucleic acid.

44-45. (canceled)

46. A method of obtaining nucleic acids from a biological sample and preparing the nucleic acids for analysis, the comprising:
placing a biological sample comprising nucleic acids in a vessel to form a sample mixture,
adding a magnetic nanoparticle composition to the sample mixture, the magnetic particle composition comprising a plurality of magnetic nanoparticles having a core and a coating on the core, the coating having a thickness of from about 1.5 nm to about 2 nm;
mixing the sample mixture and the magnetic nanoparticles and incubating for an incubation period;
separating the magnetic nanoparticles from the sample mixture;
adding an elution medium to the vessel and mixing the magnetic nanoparticles with the elution medium; and
removing the eluate from the vessel.

47. The method of claim 46, further comprising contacting the biological sample with guanidine thiocyanate (GTC) and Proteinase K to form the sample mixture;
incubating the sample mixture at an elevated temperature for a sample preparation period; and
adding neat alcohol to the sample mixture in the vessel.

48. The method of claim 46, wherein the magnetic nanoparticles are separated from the sample mixture by:
applying a magnetic force to separate the magnetic nanoparticles from a supernatant within the vessel;
removing the supernatant from the vessel without disturbing the separating magnetic nanoparticles; and
washing the separated magnetic nanoparticles one or more times with an alcohol solution.

49. The method of claim 46, wherein the elution period is less than 10 minutes.

50. (canceled)

51. The method of claim 46, wherein the sample preparation period and/or the incubation period is about 10 minutes or less.

52. The method of claim 46, wherein the alcohol solution comprises about 80% v/v or greater of alcohol.

53. The method of claim 46, wherein the nucleic acids are RNA or DNA.

54. (canceled)
